SEQUENCE LISTING (58 sequences)

SEQ ID NO: 1 - protein, 16 aa, Mus musculus
EPRGPTIKPSPPCKCP

SEQ ID NO: 2 - protein, 15 aa, Homo sapiens
EPKSSDKTHTCPPCP

SEQ ID NO: 3 - protein, 217 aa, Mus musculus
APNLLGGPSVFIFPPKIKDVLMISLSPMVTCVVVDVSEDDPDVQISWFVNNVEVLTAQTQTHREDYQSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 217 aa, Homo sapiens
APELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 11 aa, Artificial Sequence
  Protein purifcation tag
DPGHHHHHHSS

SEQ ID NO: 6 - protein, 13 aa, Artificial Sequence
  Protein purification tag
DPGWSHPQFEKSS

SEQ ID NO: 7 - protein, 40 aa, Artificial Sequence
  Artificially designed peptide spacer sequence
DPGWSHPQFEKSSGGGGSGGGGSGGGGGSSWSHPQFEKSS

SEQ ID NO: 8 - protein, 14 aa, Artificial Sequence
  Protein purification tag with C-terminal cysteine residue
DPGWSHPQFEKRSC

SEQ ID NO: 9 - protein, 18 aa, Artificial Sequence
  Protein purification tag
DPGLNDIFEAQKIEWHEV

SEQ ID NO: 10 - protein, 9 aa, Human cytomegalovirus
NLVPMVATV

SEQ ID NO: 11 - protein, 9 aa, Homo sapiens
SLLMWITQV

SEQ ID NO: 12 - protein, 9 aa, Homo sapiens
LMLGEFLKL

SEQ ID NO: 13 - protein, 9 aa, Human herpesvirus 4
GLCTLVAML

SEQ ID NO: 14 - protein, 9 aa, Influenza A virus
GILGFVFTL

SEQ ID NO: 15 - protein, 9 aa, Human immunodeficiency virus
SLYNTVATL

SEQ ID NO: 16 - protein, 9 aa, Human papillomavirus type 16
YMLDLQPET

SEQ ID NO: 17 - protein, 10 aa, Homo sapiens
ELAGIGILTV

SEQ ID NO: 18 - protein, 9 aa, Homo sapiens
ILAKFLHWL

SEQ ID NO: 19 - protein, 9 aa, Homo sapiens
YLSGANLNL

SEQ ID NO: 20 - protein, 9 aa, Homo sapiens
LLGRNSFEV

SEQ ID NO: 21 - protein, 9 aa, Homo sapiens
KIFGSLAFL

SEQ ID NO: 22 - protein, 9 aa, Homo sapiens
SLSKILDTV

SEQ ID NO: 23 - protein, 9 aa, Homo sapiens
STAPPVHNV

SEQ ID NO: 24 - protein, 9 aa, Homo sapiens
YVDPVITSI

SEQ ID NO: 25 - protein, 10 aa, Homo sapiens
KLVVVGAGGV

SEQ ID NO: 26 - protein, 10 aa, Homo sapiens
KLVVVGAVGV

SEQ ID NO: 27 - protein, 10 aa, Homo sapiens
KLVVVGADGV

SEQ ID NO: 28 - protein, 10 aa, Homo sapiens
KLVVVGAGDV

SEQ ID NO: 29 - protein, 9 aa, Homo sapiens
YMDGTMSQV

SEQ ID NO: 30 - protein, 16 aa, Artificial Sequence
  Artificially designed linker sequence
GCGSGGGGAPGSGGGS

SEQ ID NO: 31 - protein, 288 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIP

SEQ ID NO: 32 - protein, 283 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCNVGSDWRFLRGYHEYAYDGKDYIALKEDLRSWTAAEMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIP

SEQ ID NO: 33 - protein, 675 aa, Artificial Sequence
  Fusion protein sequence designed for combination with antigen pepetide sequence to function as aAPM
GCGSGGGGAPGSGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMRSASGGGGSGGGGSGGGGSASGGGGGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCNVGSDWRFLRGYHQYAYDGKDYIALKEDLRSWTAAEMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPTSQLTSEPRGPTIKPSPPCKCPAPNLLGGPSVFIFPPKIKDVLMISLSPMVTCVVVDVSEDDPDVQISWFVNNVEVLTAQTQTHREDYQSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDPGWSHPQFEKSS

SEQ ID NO: 34 - protein, 99 aa, Homo sapiens [sequence omitted]

SEQ ID NO: 35 - protein, 25 aa, Artificial Sequence
  artificially designed linker sequence
RSASGGGGSGGGGSGGGGSASGGGG

SEQ ID NO: 36 - protein, 283 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCNVGSDWRFLRGYHQYAYDGKDYIALKEDLRSWTAAEMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIP

SEQ ID NO: 37 - protein, 6 aa, Artificial Sequence
  Artificially designed linker sequence
TSQLTS

SEQ ID NO: 38 - protein, 233 aa, Mus musculus
EPRGPTIKPSPPCKCPAPNLLGGPSVFIFPPKIKDVLMISLSPMVTCVVVDVSEDDPDVQISWFVNNVEVLTAQTQTHREDYQSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - protein, 18 aa, Influenza A virus
MAKANLLVLLCALAAADA

SEQ ID NO: 40 - protein, 18 aa, Homo sapiens
MKWVTFISLLFLFSSAYS

SEQ ID NO: 41 - protein, 13 aa, Human cytomegalovirus
YQEFFWDANDIYR

SEQ ID NO: 42 - protein, 13 aa, Mycobacterium tuberculosis
MAKTIAYDEEARR

SEQ ID NO: 43 - protein, 20 aa, Homo sapiens
DNKSKITIDIHFLERKMQHH

SEQ ID NO: 44 - protein, 13 aa, Influenza A virus
PKYVKQNTLKLAT

SEQ ID NO: 45 - protein, 12 aa, Clostridium tetani
GQIGNDPNRDIL

SEQ ID NO: 46 - protein, 13 aa, Human herpesvirus 4
KELKRQYEKKLRQ

SEQ ID NO: 47 - protein, 18 aa, Artificial Sequence
  Artificially designed protein linker
GSGGGGSGGGGSGGGGSG

SEQ ID NO: 48 - protein, 15 aa, Homo sapiens
PVSKMRMATPLLMQA

SEQ ID NO: 49 - protein, 16 aa, Artificial Sequence
  Artificially designed peptide linker sequence
GSGGSLVPRGSGGSGS

SEQ ID NO: 50 - protein, 198 aa, Homo sapiens
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 51 - protein, 192 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEN

SEQ ID NO: 52 - protein, 25 aa, Homo sapiens
MAISGVPVLGFFIIAVLMSAQESWA

SEQ ID NO: 53 - protein, 30 aa, Homo sapiens
KQLKKKLQALKKKNAQLKWKLQALKKKLAQ

SEQ ID NO: 54 - protein, 30 aa, Homo sapiens
KQLEKELQALEKENAQLEWELQALEKELAQ

SEQ ID NO: 55 - protein, 493 aa, Artificial Sequence
  Artificially designed fusion protein
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKNSGGGGSKQLKKKLQALKKKNAQLKWKLQALKKKLAQASEPRGPTIKPSPPCKCPAPNLLGGPSVFIFPPKIKDVLMISLSPMVTCVVVDVSEDDPDVQISWFVNNVEVLTAQTQTHREDYQSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDPGLNDIFEAQKIEWHEHHHHHH

SEQ ID NO: 56 - protein, 503 aa, Artificial Sequence
  Artificially designed fusion protein
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENSGGGGSKQLEKELQALEKENAQLEWELQALEKELAQASEPRGPTIKPSPPCKCPAPNLLGGPSVFIFPPKIKDVLMISLSPMVTCVVVDVSEDDPDVQISWFVNNVEVLTAQTQTHREDYQSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDPGWSHPQFEKSSGGGGSGGGGSGGGGGSSWSHPQFEKSS

SEQ ID NO: 57 - DNA, 2115 bp, Artificial Sequence
  Artificially designed nucleotide sequence encoding fusion protein/aAPM
atggccaaggccaacctgctcgtgctgctctgcgccctggcggccgctgacgccaacctggtgcccatggtggccaccgtgggctgcggatccggaggaggcggcgcgccggggtctggaggtggatccatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgagatcggctagtggtggtggtggttctggtggtggtggttctggtggtggtggttctgctagcggcggaggcggctcccactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccagagtattgggacggggagacacggaaagtgaaggcccactcacagactcaccgagtggacctggggaccctgcgcggctgttataaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccactagccaattgactagtgagcccagaggacccacaatcaagccctctcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattaccagagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaggatcccgggtggagccaccctcagttcgagaagtctagagggcccgtttaa

SEQ ID NO: 58 - DNA, 3273 bp, Artificial Sequence
  Artificially designed nucleotide sequence encoding fusion protein/aAPM
atgaagtgggtgaccttcatcagcctgctgttcctgttctcgagcgcttactccatggctaagacaatcgcttacgatgaggaagccagacggggttccggaggaggcgggagcggaggcggagggagtggaggcggcggatccggcggggacaccagaccacgtttcttggagtactctacgtctgagtgtcatttcttcaatgggacggagcgggtgcggtacctggacagatacttccataaccaggaggagaacgtgcgcttcgacagcgacgtgggggagttccgggcggtgacggagctggggcggcctgatgccgagtactggaacagccagaaggacctcctggagcagaagcggggccgggtggacaactactgcagacacaactacggggttgtggagagcttcacagtgcagcggcgagtccatcctaaggtgactgtgtatccttcaaagacccagcccctgcagcaccataacctcctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagaattccggcggaggcggatccaagcagctgaagaaaaagctccaggccctgaagaagaaaaacgcccagctcaagtggaagctccaggccctgaaaaagaagctggcccaggctagcgagcccagagggcccacaatcaagccctctcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattaccagagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaggatcccgggctgaacgacatcttcgaggctcagaaaatcgaatggcacgagcaccatcaccatcaccatggctccggcgagggcagaggaagcttgctgacatgtggcgacgtggaagagaaccctggccctatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaattccggcggaggcggatccaagcagctcgaaaaggagctgcaagccctggagaaggagaacgcccagctggaatgggaactccaggccctggagaaagagctggcccaggctagcgagcccagagggcccacaatcaagccctctcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattaccagagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaggatcccgggtggagccaccctcagttcgagaagtctagtggcggaggaggctccggaggcggaggcagcggcggaggaggcggatctagttggagccaccctcagttcgagaaggtctag

(19) United States
(12) Patent Application Publication
(10) Pub. No.: US 2022/0381770 A1
(43) Pub. Date: Dec. 1, 2022

(54) ARTIFICIAL ANTIGEN PRESENTING MOLECULES AND THEIR USES

(71) Applicant: UNIVERSITAET HEIDELBERG, Heidelberg (DE)

(72) Inventors: Frank MOMBURG, Heidelberg (DE); Marten MEYER, Heidelberg (DE); Inka ZOERNIG, Frankfurt (DE); Dirk JAEGER, Konstanz (DE); Niels HALAMA, Seeheim-Jugenheim (DE); Iris KAISER, Weinheim (DE)

(73) Assignee: UNIVERSITAET HEIDELBERG, Heidelberg (DE)

(21) Appl. No.: 17/770,877

(22) PCT Filed: Oct. 23, 2020
(86) PCT No.: PCT/EP2020/079924

§ 371 Date: Apr. 21, 2022

(30) Foreign Application Priority Data
Oct. 29, 2019 (EP) 19205926.9

(51) Int. Cl. G01N 33/50; C07K 16/24; C07K 16/28; C12N 5/0783

(52) U.S. Cl. CPC G01N 33/505; C07K 16/249; C07K 16/2818; C12N 5/0636; A61K 2039/5158

(57) ABSTRACT

The present invention relates to artificial Antigen Presenting Cells (aAPCs) comprising artificial Antigen Presenting Molecules (aAPMs) and, in particular, comprising dimers of the aAPMs as well as to methods for producing aAPCs. The invention further relates to compositions comprising the aAPCs and to vectors encoding the aAPMs of the aAPCs. Embodiments of the invention have been particularly developed for use in assays for determining an antigen-specific T cell response or a plurality of antigen-specific T cell responses and will be described hereinafter with reference to this application. However, it will be appreciated that the invention is not limited to this particular field of use.

### FIELD OF THE INVENTION

The present invention relates artificial Antigen Presenting Cells (aAPCs) comprising artificial Antigen Presenting Molecules (aAPMs) and, in particular, comprising dimers of the aAPMs as well as to methods for producing aAPCs. The invention further relates to compositions comprising the aAPCs and to vectors encoding the aAPMs of the aAPCs. Embodiments of the invention have been particularly developed for use in assays for determining an antigen-specific T cell response or a plurality of antigen-specific T cell responses and will be described hereinafter with reference to this application. However, it will be appreciated that the invention is not limited to this particular field of use.

### BACKGROUND OF THE INVENTION

Any discussion of the background art throughout the specification should in no way be considered as an admission that such art is widely known or forms part of common general knowledge in the field.

During an immune response, the presentation of antigenic peptides to antigen-specific T cells typically occurs through antigen presenting molecules (APMs), namely through antigenic peptides being complexed with major histocompatibility complexes (MHCs), positioned on the outer membrane of an antigen presenting cell (APC). APCs are distinguished by the class of MHCs that they utilise for presenting an antigenic peptide. Most cells in the body can present antigenic peptides via MHC class I molecules to CD8+ cytotoxic T cells. Antigenic peptides presented by MHC class I molecules are typically derived from cytosolic proteins. However, “specialised” or “professional” APCs are those, which present antigenic peptides via MHC class I or MHC class II molecules to cytotoxic T cells or to CD4+ helper T cells, respectively. Antigenic peptides presented by MHC class II molecules are typically derived from extracellular proteins.

Artificial antigen presenting cells (aAPCs) have been developed in an attempt to generate large numbers of functional antigen-specific T cells in vitro for subsequent use in therapy. In addition, aAPCs can be used to study antigen-specific T cell responses and assays for the assessment of T cell recognition of the disease-associated antigens prior and during immunotherapy utilising aAPCs are available. Various types of aAPCs are known, which utilise membrane-bound APMs such as MHC class I molecules. Some aAPCs utilise synthetic vesicles or liposomes comprising lipid bilayers in conjunction with membrane-bound APMs. Recombinantly engineered aAPCs are also known, for example, mouse fibroblasts transfected with vector constructs for the expression of specific peptide-loaded MHCs have been described. In addition, micro- and nanoparticle systems have been developed, in which the particles are loaded with APMs. However, in such systems the choice of APM is limited due to structural compatibility with the trans-membrane domains allowing for stable attachment to the particles, while allowing for specific and stable binding to the target T cells to reproducibly elicit an antigen-specific T cell response.

In addition, artificial APMs (aAPMs) both as soluble analogues of proteins involved in triggering and/or attenuating immune responses as well as for use with aAPCs have been developed.

U.S. Pat. No. 6,268,411 B1 for example describes the use of soluble, multivalent peptide-loaded MHC/Ig molecules to detect, activate or suppress antigen-specific cell-dependent immune responses. The molecules described are chimeric molecules comprising an MHC class I molecule portion fused to an immunoglobulin heavy chain. In addition, covalently binding the antigenic peptide to the MHC class I molecule portion by a peptide tether is described. As such an immunoglobulin comprising heavy and light chains is described, in which the antigen-presenting MHC class I molecule portion is fused to the N-terminus of both heavy chains. While these chimeric molecules are soluble, due to the fusion of the entire MHC class I molecule portion to an immunoglobulin comprising two heavy and two light chains, they are certainly complex molecules and their suitability for use with aAPCs is not discussed.

There is a need in the art for improved and versatile tools for use in assays for the determination of antigen-specific T cell responses. Such tools include innovative aAPCs comprising aAPMs specifically designed to stably bind an antigenic peptide while being attached to the surface of the aAPC such as to reproducibly elicit a measurable T cell response when brought in contact with a population of T cells.

It is an object of the present invention to overcome or ameliorate at least one of the disadvantages of the prior art, or to provide a useful alternative. In particular, it is an object of the present invention to provide improved aAPCs comprising aAPMs specifically designed to stably bind an antigenic peptide while being attached to the surface of the aAPC such as to elicit a measurable T cell response when brought in contact with a population of T cells. In particular, it is an object of the present invention to provide improved aAPCs and aAPMs improving the versatility of assays for determining disease-associated, antigen-induced T cell responses.

### SUMMARY OF THE INVENTION

As indicated above, the present invention aims at providing aAPCs for use in versatile assays of eliciting and determining T cell immune responses to selected antigens. In particular, the assessment of disease-associated antigen recognition by patient's T cells will allow clinically relevant approaches to personalise therapy. In light of the limited patient sample available in a clinical setting, the aAPCs suitable for an assessment of a complex immune response are preferably configured such as to not only present an antigen in order to elicit an immune response by a T cell but also to detect and/or capture effector molecules specifically released by the T cell in response to antigen presentation.

Accordingly, in a first aspect, the present invention relates to an artificial antigen-presenting cell (aAPC) for the detection of effector molecules of a T cell in response to presentation of an antigen peptide sequence, the aAPC comprising:
(a) a surface, wherein the surface is the surface of a particle, optionally the surface is the surface of a bead;
(b) one or more artificial Antigen Presenting Molecules (aAPMs), wherein the aAPMs are attached to the surface via attachment sequences or via direct chemical conjugation; and
(c) one or more capture molecules attached to the surface,

wherein each of the one or more aAPMs and/or each of the one or more dimers comprises an identical antigen peptide sequence.

Different types of particles may serve to provide the surface of the aAPCs of the present invention, i.e. to provide the surface to which the one or more aAPMs and the one or more capture molecules are attached. In particular, rigid spherical particles, non-spherical particles and/or fluidic lipid bilayer-containing systems are all particles suited for providing the surface of the aAPCs of the present invention. For example, polystyrene latex microbeads, magnetic nano- and micro-particles or beads, nano-sized quantum dots and poly(lactic-co-glycolic acid) (PLGA) microspheres are known rigid spherical particles suitable for use in the aAPCs of the present invention, while carbon nanotube bundles, ellipsoid PLGA microparticles, and nanoworms are known non-spherical particles suitable for use in the aAPCs of the present invention and 2D-supported lipid bilayers (2D-SLBs), liposomes and RAFTsomes/microdomain liposomes and SLB particles are known fluidic lipid bilayer-containing systems suitable for use in the aAPCs of the present invention.

The aAPCs of the present invention provide a robust, easy-to-use, cost and time effective multiplex platform to detect a variety of many antigen-specific T cells from a sample by utilising custom-made aAPC compositions tailored for multiplexed assessment of T cell responses.

The aAPMs of the present invention, i.e. aAPMs that must be attachable to the surface of an aAPC, may be any monomeric, dimeric or multimeric molecules comprising an MHC portion configured to elicit a T cell response, i.e. to activate a T cell. For example, the aAPM may be: a monomeric, dimeric or multimeric molecule comprising soluble antigenic peptide-loadable MHC class I or soluble antigenic peptide-loadable MHC class II portions; a molecule comprising an antigenic peptide covalently linked to and, preferably, at the same time trapped in an antigen-presenting domain. As such, the aAPM may be a tagged recombinant soluble MHC-I molecule assembled with β₂-microglobulin and a synthetic peptide, or a tagged recombinant soluble MHC-I molecule covalently linked with β₂-microglobulin and assembled with a synthetic peptide. Notwithstanding, the antigen presenting domain of the aAPMs of the present invention may comprise any human leukocyte antigen (HLA) allele known to the skilled person. A database listing substantially all known HLA alleles as named in the World Health Organization Nomenclature Committee Reports is available at: https://www.ebi.ac.uk/ipd/imgt/hla/allele.html. In addition, aAPMs of the present invention are versatile with respect to their potential attachment to the surface of an aAPC. However, the versatility is particularly improved as the aAPMs of the present invention allow for antigen-specific stimulation of an immune response of either CD8+ and/or CD4+ T cells.

Preferably, the aAPMs of the aAPCs of the present invention consist of (a) a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigen peptide sequence; or (b) a single polypeptide sequence comprising in amino-to-carboxy terminal order: an antigen-presenting domain and an attachment sequence, wherein the sequence of the antigen presenting domain comprises an N-terminal antigen peptide sequence. As such, rather than being assembled from various separately produced polypeptide chains, the aAPMs described here are produced as single polypeptide chains, which already comprise all domains and sequences required for the aAPM to function in the aAPCs of the present invention, in particular the so-produced single polypeptide sequence also comprises the antigen peptide sequence to be presented. As such, subsequent loading or linking with the antigen peptide sequence is not required. This avoids the potential inefficiencies and inconsistencies between producing batches of aAPMs being inherent in subsequent antigen peptide loading or linking procedures previously described.

Furthermore, the aAPCs of the present invention may comprise aAPMs in dimeric form. Specifically, the aAPCs of the present invention may comprise a dimer comprising an aAPM, wherein
(d) the dimer is a homodimer or heterodimer of two aAPMs, or
(e) the dimer is a heterodimer of an aAPM and a second molecule.

As such, in a second aspect, the present invention relates to a composition comprising
(a) a plurality of aAPCs according to the first aspect, wherein the composition comprises a plurality of identical aAPCs, optionally the identical aAPCs comprise a single capture molecule specific for a respective single effector molecule or the identical aAPCs comprise several capture molecules specific for several respective effector molecules, or
(b) a plurality of groups of aAPCs according to the first aspect, wherein all aAPCs of each group are coded identically and wherein the antigen peptide sequence presented by the aAPCs of each group is identical within the group but different between each of the groups, optionally the aAPCs of each group comprise a single capture molecule specific for a respective single effector molecule or the aAPCs of each group comprise several capture molecules specific for several respective effector molecules.

The aAPMs, aAPCs and compositions comprising the same of the present invention are particularly useful in various embodiments of multiplexed assays for the assessment of antigen-specific T cell responses. Specifically, the aAPMs, aAPCs and compositions comprising the same of the present invention can be composed such as to provide for various levels of complexity, i.e. various degrees of “multiplexicity”.

Therefore, in a third aspect, the present invention relates to an assay for determining an antigen-specific T cell response, the assay comprising the following steps:
(a) contacting T cells with a composition according to the second aspect under conditions and for a time suitable to elicit a response from the T cells upon presentation of the antigen peptide sequence by the identical aAPCs;
(b) separating the aAPCs from the T cells;
(c) contacting the aAPCs with detection antibodies against one or more effector molecules for which the one or more capture molecules of the aAPCs are specific;
(d) analysing the release of effector molecules of the T cells by detecting the effector molecules captured by the one or more capture molecules of the aAPCs,

thereby determining the T cell response specific to the antigen peptide sequence presented by the identical aAPCs.

In a fourth aspect, the present invention relates to an assay for determining a plurality of antigen-specific T cell responses, the assay comprising the following steps:
(a) contacting T cells with a composition according to the second aspect under conditions and for a time suitable to elicit responses from the T cells upon presentation of each of the different antigen peptide sequences presented by each group of aAPCs;
(b) separating the aAPCs from the T cells;
(c) contacting the aAPCs with detection antibodies against one or more effector molecules for which the one or more capture molecules of the aAPCs within each group of aAPCs are specific;
(d) identifying and separating the aAPCs of each of the groups of aAPCs;
(e) analysing the release of effector molecules of the T cells by detecting the effector molecules captured by the one or more capture molecules of the aAPCs within each group of aAPCs,

thereby determining a plurality of antigen-specific T cell responses specific to each antigen peptide sequence presented by each group of aAPCs.

In a fifth aspect, the present invention relates to a vector comprising
(a) a polynucleotide sequence encoding the single polypeptide sequence of the aAPM as defined for the first aspect, or
(b) a polynucleotide sequence for polycistronic expression of both peptide chains of a dimer as defined for of the first aspect.

In a further aspect, the present invention relates to a method of manufacturing an aAPC according to the first aspect, wherein the method comprises covalently attaching
(a) an aAPM, preferably an aAPM is a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigen peptide sequence, and
(b) a capture molecule, preferably a capture antibody, to the surface of a microsphere, preferably a colour-coded microsphere.

### FIGURES

Embodiments of the invention will now be described, by way of example only, with reference to the accompanying drawings in which:

FIG. 1 illustrates the domain structure and assembly of an exemplary MHC-I dimer (aAPM) for use in the aAPCs of the present invention;

FIG. 2 illustrates the domain structure and assembly of an exemplary MHC-II dimer (aAPM) for use in the aAPCs of the present invention

FIG. 3 illustrates the domain structure of exemplary polycistronic MHC-I aAPM construct and its assembly into an MHC-I aAPM for use in the aAPCs of the present invention.

FIG. 4 illustrates the domain structure of a further MHC-I aAPM construct and its assembly into an MHC-I aAPM for use in the aAPCs of the present invention.

FIG. 5 illustrates the assembly of an aAPC of the present invention for use in a multiplex assay for the determination of an antigen-specific T cell response in accordance with the assays and methods described. (a) aAPC concept; (b) Architecture and assembly of an exemplary aAPC of the present invention; (c) aAPC co-coordinate system; (d) Basic principle of a multiplex assay.

FIG. 6 illustrates two differing exemplary T-Plex Assay workflows. (a) T-Plex Assay workflow based on bead spotting followed by orbital shaking; (b) T-Plex “rotation-one-tube-reaction” principle.

FIG. 7 illustrates the assembly of a further aAPC of the present invention for use in a multiplex assay for the determination of an antigen-specific T cell response in accordance with the assays and methods described:

FIG. 8 illustrates exemplary assembly concepts for the design of CD4+ and/or CD8+ T-Plex² assays in accordance with the assays and methods described.

FIG. 9 illustrates the T-Plex Assay proof-of-concept.

FIG. 10 shows that bystander T cells do not decrease the sensitivity of the sensitivity of the T-Plex Assay, as is further described in Example 2 below.

FIG. 11 illustrates the comparison of pMHC-I multimer staining and the assays of the present invention in accordance with the experiments of Example 3.

FIG. 12 illustrates a first set of experiments performed to optimize T-Plex Assay parameters, as is further described in Example 4 below.

FIG. 13 illustrates a second set of experiments performed to optimize T-Plex Assay parameters as described in Example 4 below.

FIG. 14 illustrates a number of T-Plex bead assembly variations and their impact on T-Plex Assay performance, as is further described in Example 5 below.

FIG. 15 shows the antigen-specific detection of MTB/DR3 CD4⁺ T cell clone RP15.1.1 by pMHC-II-Fc loaded T-Plex beads, as is further described in in Example 6 below.

FIG. 16 illustrates proof-of-principle experiments for a T-Plex² Assay for the antigen-specific detection of CD4⁺ T cells, as is further described in Example 7 below.

FIG. 17 illustrates antigen-specific T cell detection using the T-Plex Assay does not change the phenotype of the original sample, as is further described in Example 8 below.

FIG. 18 illustrates the successful eukaryotic cell-based production and antigen-specific binding of soluble pMHC-I-Fc aAPMs, as is further described in Example 9 below.

FIG. 19 illustrates the successful production and antigen-specific binding validation of soluble pMHC-II-Fc aAPMs, as is further described in Example 10 below.

### DETAILED DESCRIPTION OF THE INVENTION

In order to provide a clear and consistent understanding of the specification and claims, and the scope to be given such terms, the following definitions are provided.

### Definitions

In the context of the present application, the below-defined terms shall be understood to mean the following:

“artificial Antigen Presenting Cell (aAPC)”—an artificially generated surface, including the surface of a living cell or of a synthetic material configured to bind an artificial Antigen Presenting Molecule (aAPM; as directly defined below) including peptide-loaded MHC molecules, such as to present an antigen suitable to stimulate a respective antigen-specific T cell leading to a similar stimulation as occasioned by naturally occurring antigen-presenting cells. As an example, a particle, which has a plurality of defined aAPMs attached to its surface constitutes an aAPC according to the present disclosure. Further, in the context of the present application, an aAPC can additionally be configured, i.e. have the capacity, to capture one or more effector molecules secreted by an activated T cell. Different types of particles may serve to provide the surface of the aAPCs of the present invention, i.e. to provide the surface to which the one or more aAPMs and the one or more capture molecules are attached.

In particular, rigid spherical particles, non-spherical particles and/or fluidic lipid bilayer-containing systems are all particles suited for providing the surface of the aAPCs of the present invention. For example, polystyrene latex microbeads, magnetic nano- and micro-particles or beads, nano-sized quantum dots and poly(lactic-co-glycolic acid) (PLGA) microspheres are known rigid spherical particles suitable for use in the aAPCs of the present invention, while carbon nanotube bundles, ellipsoid PLGA microparticles, and nanoworms are known non-spherical particles suitable for use in the aAPCs of the present invention and 2D-supported lipid bilayers (2D-SLBs), liposomes and RAFTsomes/microdomain liposomes and SLB particles are known fluidic lipid bilayer-containing systems suitable for use in the aAPCs of the present invention. In particular embodiments, a bead-based aAPC as defined here is referred to as a “T-Plex bead” below.

“artificial Antigen Presenting Molecule (aAPM)”—a soluble or cell membrane-associated recombinantly-produced MHC protein comprising an antigen presenting domain and a specific and well-defined attachment sequence. For example, the MHC protein may be loadable with or already loaded with an antigenic peptide, such that the aAPM can mediate binding of the aAPM to a T cell receptor with the matching (cognate) antigen-specificity, while the attachment sequence is configured to immobilize the aAPM on the surface of a living cell or of a synthetic material such as, e.g. of a polystyrene-based microsphere or bead.

“antigen presenting domain”—the protein domain of an aAPM (as defined directly above), which is configured to be loadable with or which is loaded with an antigenic peptide to be presented and where the presentation can mediate the binding of a T cell receptor with the matching (cognate) antigen-specificity. In some embodiments described in the present application, the antigen presenting domain of the aAPM is or is derived from: a peptide-loaded, β₂-microglobulin-associated MHC-I ectodomain encoded by allelic variants of HLA-A, B, C, E or F genes (or respective polymorphic MHC-I genes from non-human species); or a peptide-loaded MHC-II ectodomain encoded by allelic variants of HLA-DRB genes and HLA-DRA, or allelic variants of HLA-DQA/HLA-DQB genes, or allelic variants of HLA-DPA/DPB genes (or respective polymorphic MHC-II genes from non-human species). “derived from” in this context means that a domain, which is “derived from” a first domain must have at least 80% sequence identity with this first domain and, importantly, must still be able to mediate the binding of a T cell receptor with the matching (cognate) antigen-specificity.

“dimerization domain”—a protein domain in a monomeric peptide chain configured to bind a corresponding protein domain in a separate monomeric peptide chain, wherein binding of the two corresponding protein domains leads to the formation of a dimeric molecule comprising both peptide chains. For example, the hinge domain of immunoglobulin G (IgG) or a heterophilic parallel coiled-coil leucine zipper sequence or a combination of both can act as dimerization domains in the aAPMs described in the present disclosure.

“Ig Fc domain”—a protein sequence, which consists of or is closely derived from the constant heavy chain (CH) domains 2 and 3 (CH2 and CH3) of IgG. “derived from” in this context means that a sequence, which is “derived from” a first sequence consisting of the constant heavy chain (CH) domains 2 and 3 (CH2 and CH3) of IgG must have at least 80% sequence identity with this first sequence and, importantly, must still be functionally equivalent to the first sequence.

“attachment sequence”—a peptide sequence of the aAPM configured to mediate the attachment of the aAPM to the surface of a living cell or of a synthetic material such as, e.g. of a polystyrene-based microsphere or bead. In particular embodiments, the attachment sequence may be part of the Ig Fc domain or may be a short-peptide sequence such as a polyhistidine-tag, Strep-tag, biotinylated AviTag that facilitates affinity-chromatography-based protein purification as well as binding of a soluble aAPM to the surface of an aAPC.

“MHC class I portion”—peptide-loadable MHC-I heavy chain ectodomain (α₁-α₃) associated with β₂-microglobulin.

“MHC class II portion”—heterodimeric peptide-loadable MHC-II alpha chain ectodomain (α₁-α₂) associated with an MHC-II beta chain (β₁-β₂) ectodomain.

“effector molecule”—a T cell-secreted molecule, which has stimulatory or inhibitory immune functions such as a cytokine, perforin or an enzyme such as granzyme B eliciting cytotoxicity.

“capture molecule”—an antibody or recombinant receptor protein that specifically binds to and thereby captures a defined effector molecule

“costimulatory-acting molecule”—an agonistic antibody or recombinant soluble ligand that that engages a T cell-costimulatory receptor, e.g. CD28 or 4-1BB.

“multiplexation”—refers to a methodological process that allows the simultaneous yet differential detection of multiple analytes in a single reaction, hence an experimental assay or method relying on this process may be termed “multiplex assay” in this disclosure. In the context of this disclosure the term “multiplexation” is typically used to describe the simultaneous detection of multiple antigen-specific T cell populations (analytes) through the usage of identifiable aAPCs (such as fluorescently colour-coded “barcoded” aAPCs) with effector molecule capture capacity.

“T-Plex Assay”—an assay that allows the detection of multiple antigen-specific T cell populations on the basis of separately identifiable populations of T-Plex beads being utilized (one-dimensional multiplexation). For example, a T-Plex Assay may be performed with separately identifiable populations of T-Plex beads, where each population presents a separate aAPM. If in such a T-Plex Assay all T-Plex beads have the capacity to detect at least one effector molecule (e.g. interferon-γ), a multiplexed assessment of at least two dimensions can be achieved, namely: (1) the detection of multiple antigen-specific T cell populations; as well as (2) the simultaneous assessment whether such an antigen-specific T cell population secretes the effector molecule (here interferon-γ) or not. A T-Plex Assay that allows the simultaneous detection of multiple antigen-specific T cell populations and their secreted effector molecules using T-Plex beads with the capacity to capture and assess multiple effector molecules has been termed “T-Plex² Assay”.

In addition to the above definitions, and unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Further, reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, the term “exemplary” is used in the sense of providing examples, as opposed to indicating quality. That is, an “exemplary embodiment” is an embodiment provided as an example, as opposed to necessarily being an embodiment of exemplary quality.

### DETAILED DESCRIPTION

According to the first aspect of the invention, and as also illustrated in the Figures, an artificial Antigen Presenting Cell (aAPC) comprising artificial Antigen Presenting Molecules (aAPMs) is provided.

aAPMs to be used with the aAPCs of the present invention, i.e. aAPMs which must be attachable to the surface of an aAPC, may be any monomeric, dimeric or multimeric molecule comprising an MHC portion configured to elicit a T cell response, i.e. to activate a T cell.

For example, the aAPM may be: a monomeric, dimeric or multimeric molecule comprising soluble antigenic peptide-loadable MHC class I or soluble antigenic peptide-loadable MHC class II portions; or a molecule comprising soluble MHC class I or MHC class II portions comprising an antigenic peptide covalently linked to and, preferably, at the same time trapped in an antigen-presenting domain.

Any MHC complex loaded with an antigenic peptide configured to trigger a T cell response and to activate the T cell, can be used as an aAPM once attached to the surface of an aAPC of the present invention. Such an MHC complex should satisfy at least the following criteria to in order to be configured to, i.e. be able to, to trigger a T cell response and to activate the T cell:
(1) the peptide-loaded MHC complex must have an appropriate tertiary peptide/protein structure; and
(2) the antigenic peptide is correctly presented in the antigen-presenting domain of the MHC complex.

Of course, in order to be activated by an antigenic peptide, the T cell exposed to the aAPCs of the present invention must have previously been exposed to antigenic peptides, i.e. the T-cell is not a naïve T cell.

Several different soluble MHC class I and class II molecules loaded or loadable with antigenic peptide as well as their methods of production, i.e. aAPMs suitable for use with the aAPCs of the present invention have previously been described and some of these aAPMs are commercially available.

Such known aAPMs comprising an MHC class I or MHC class II portion include aAPMs where the antigenic peptide is covalently associated with the aAPM (in case of MHC class I these are often referred to as single chain trimers (SCT aAPMs)) as well as aAPMs where the antigenic peptide is not covalently associated with the aAPM (Non-SCT aAPMs). Most commercially available aAPMs are Non-SCT aAPMs. However, the known Non-SCT aAPMs are again differentiated, namely as those which are (a) already loaded with the antigenic peptide of interest and those which (b) must subsequently be loaded with antigenic peptides of interest, i.e. they are differentiated as either being antigenic peptide-loaded or antigenic peptide-loadable MHC class I or class II aAPMs.

Monomeric non-SCT MHC class I aAPMs are typically obtained from bacterial lysates in a denatured form and are refolded into an MHC complex together with an antigenic peptide of interest and β₂-microglobulin in vitro as described by Altman et aL, (U.S. Pat. No. 5,635,363 A). As each aAPM produced in this way must be refolded in the presence of the specific antigenic peptide of interest, it is very cumbersome to produce a suite of aAPMs covering an entire antigenic peptide library. To address this issue, aAPMs, which are refolded in the presence of a photo-cleavable placeholder peptide have been described (Toebes et al., Nat. Med. 12: 246-251, 2006). In these molecules, the photo-cleavable placeholder peptide can be subsequently replaced with any antigenic peptide of interest using ultraviolet light. More recently, a process for the production of a stable refolded but “empty” MHC class I molecule, which can be loaded with an antigenic peptide of interest by use of a peptide-exchange catalyser has been described (Saini et al, Proc. Natl. Acad. Sci. 112: 202-207, 2015) and Hein et aZ, J. Cell. Sci 127: 2885-2897, 2014). However, most MHC class I aAPMs can only be produced with minimal efficiency and at extremely low yields in eukaryotic cells. The antigenic peptide-loadable aAPMs described by Schneck et al. (U.S. Pat. No. 6,268,411 B1) are produced as recombinant dimeric MHC-mIgG1 fusion constructs (full-length mouse IgG1) in a eukaryotic cell line but they are loaded with irrelevant endogenous cellular peptides during the production process. These irrelevant peptides have to be replaced with the antigenic peptides of interest in a subsequent, inefficient and often incomplete passive peptide-exchange reaction, again leading to a reduced yield of fully functional antigenic peptide-loaded aAPMs. Moreover, the production process proposed by Schneck et al. requires the establishment of an overall low efficient mouse B myeloma (J558L)-based producer cell line providing the missing murine immunoglobulin (Ig) light chain and that needs to be stably transfected with a MHC-mIgG1 construct. In addition, Schneck et al. (U.S. Pat. No. 6,268,411 B1) and Greten et al., J. Immunol. Methods 271: 125-135, 2002 reported the production of soluble dimeric SCT-mIgG1 molecules using the J558L cell line.

A soluble monomeric mouse MHC-I SCT secreted from CHO cells has been described previously (Mottez et al, J. Exp. Med. 181: 493-502, 1995). Cell membrane-associated SCT aAPMs, and in particular those that display anchoring of the covalent-associated antigenic using an additional introduced disulfide bond termed “disulfide trap” (dt) into the SCT construct design have been previously described by Hansen et al., (US 20090117153 A1). In addition, Hansen et al. reported the production of soluble monomeric disulfide trapped SCT from bacterial lysates in a denatured form and that still required an interference-prone in vitro refolding procedure prior to its usage.

Similar to the MHC class I aAPMs described above, slightly differing formats for the production of monomeric MHC class II aAPMs have been described and Vollers et al, Immunology 123:305-313, 2008, provide a useful overview. Further MHC class II aAPMs, which are produced with C-terminal dimerization domains such as leucine or Jun/Fos zippers in order to be stabilised as heterodimers, have been described.

The aAPMs of the present invention avoid these disadvantages of the prior art as they are easily produced in eukaryotic cells without the need for refolding in vitro and already contain the antigenic peptide sequence during recombinant production, in particular during recombinant production using transient-transfected mammalian expression systems such as in suspension-growing Freestyle CHO-S or Freestyle 293-F cell systems. In some embodiments, the aAPMs of the present invention can either be used as dimeric or monomeric aAPMs, but must, of course, comprise an attachment sequence for attachment to the surface of the aAPC such as to be suitable for use in the assays of the invention. A key advantage of the dtSCT-Fc construct design as proposed in this application is its capacity for efficient production of correctly folded proteins by transient-transfected mammalian expression systems such as in suspension-growing Freestyle CHO-S or Freestyle 293-F cells.

In some embodiments of the present invention, the aAPM is a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigenic peptide sequence. The antigen presenting domain may comprise any human leukocyte antigen (HLA) allele known to the skilled person. A database listing known HLA alleles as named in the World Health Organization Nomenclature Committee Reports is available at: https://www.ebi.ac.uk/ipd/imgt/hla/allele.html.

In such aAPMs, the dimerization domain can comprise an IgG hinge region. Typically, the IgG hinge region comprises of SEQ ID NO:1 or SEQ ID NO:2. The SEQ ID NO:1 is derived from the mouse IgG2a sequence and comprises a C224S mutation, while SEQ ID NO:2 is derived from the human IgG1 sequence and comprises a C220S mutation. aAPMs comprising hinge regions of SEQ ID NO:1 and SEQ ID NO:2, respectively, are complimentary such that two aAPMs having a dimerization domain comprising SEQ ID NO:1 or two aAPMs having a dimerization domain comprising SEQ ID NO:2 dimerise by forming disulfide bridges between the respective cysteine residues within the dimerization domains. As disulfide bonds are only formed between cysteines, the mutations C224S and C220S are introduced to avoid aberrant disulfides of these cysteines in the absence of Ig light chains with which they normally form intermolecular disulfide bridges.

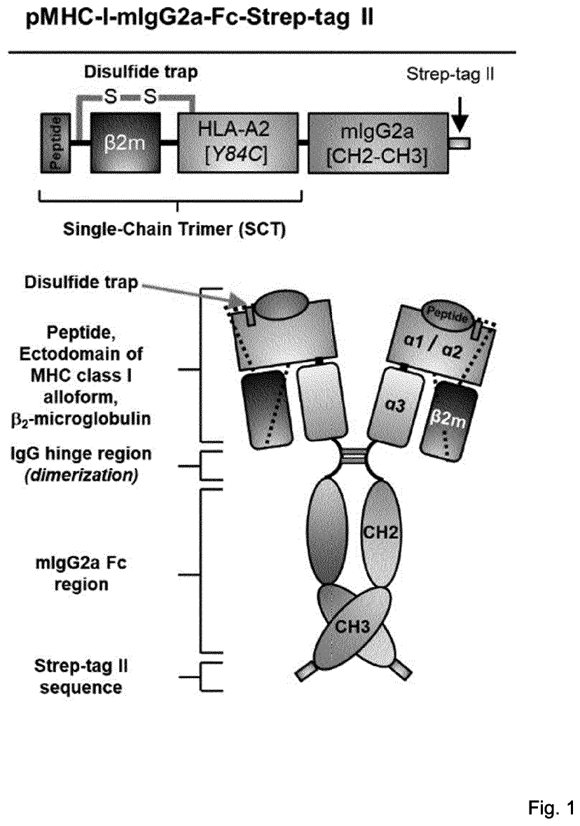

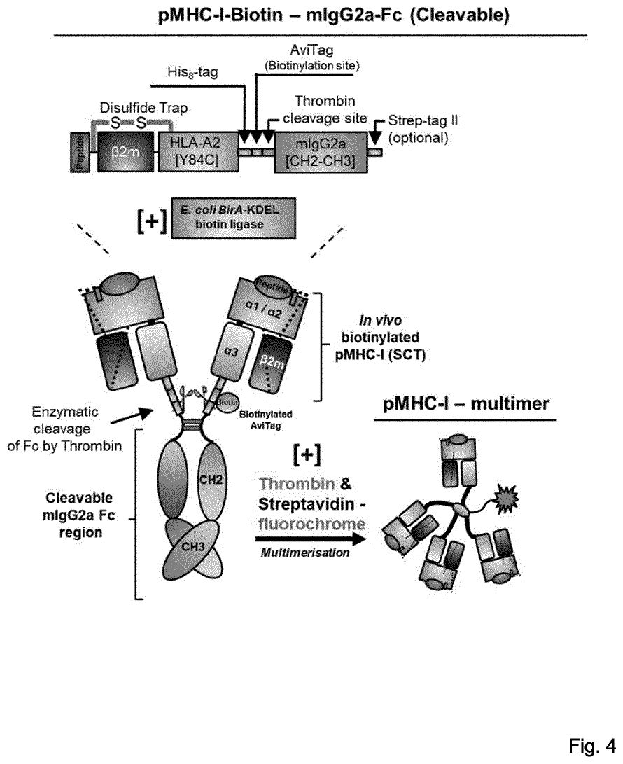

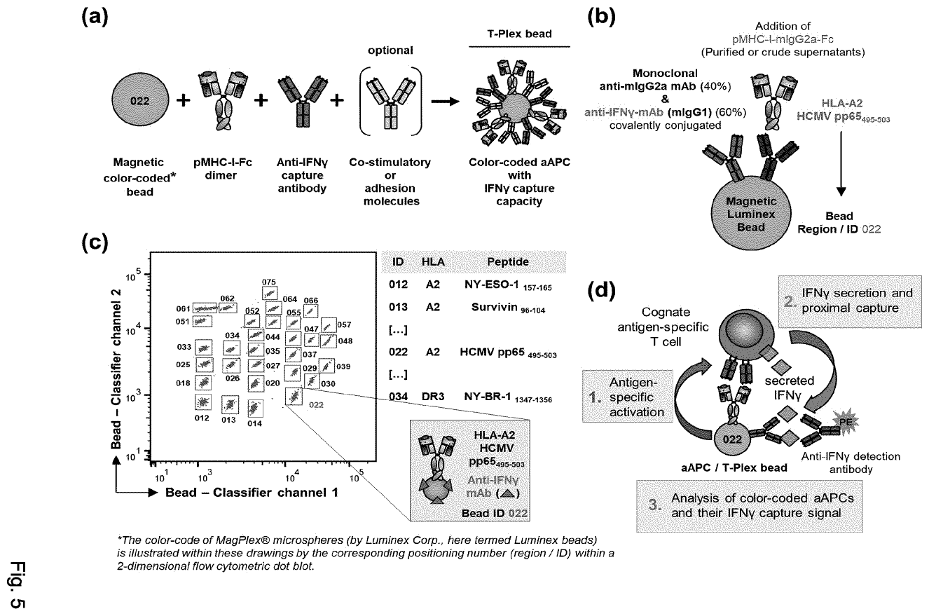

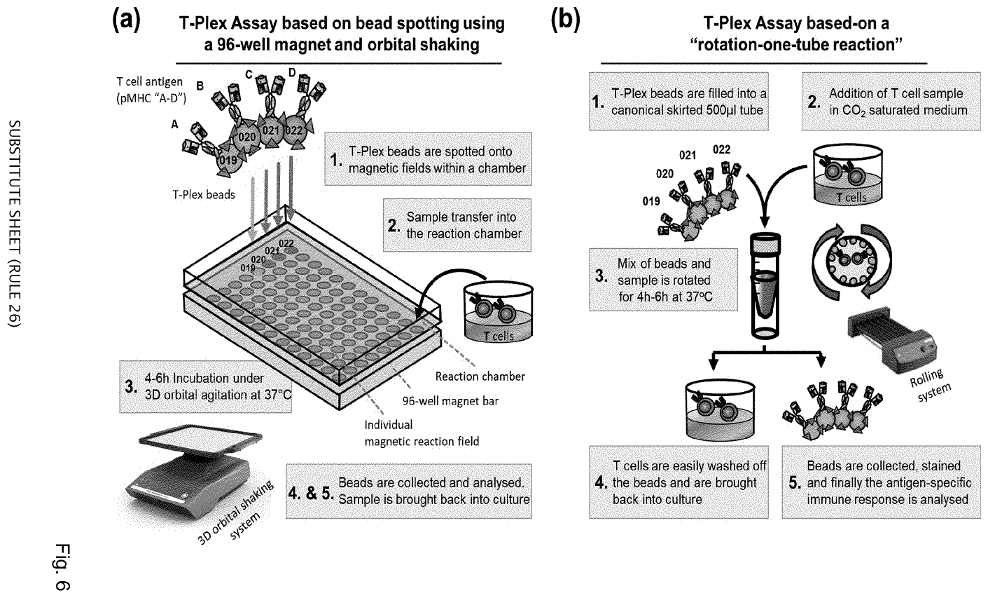

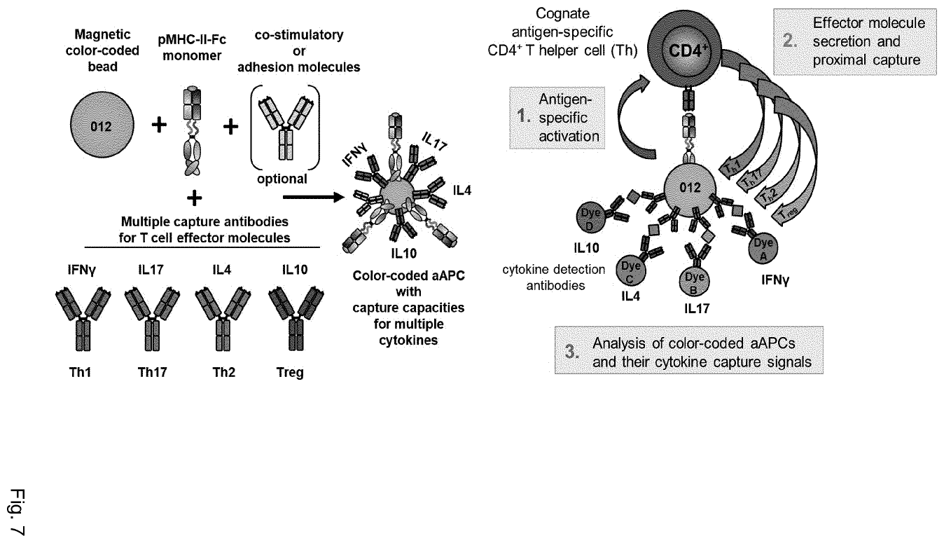

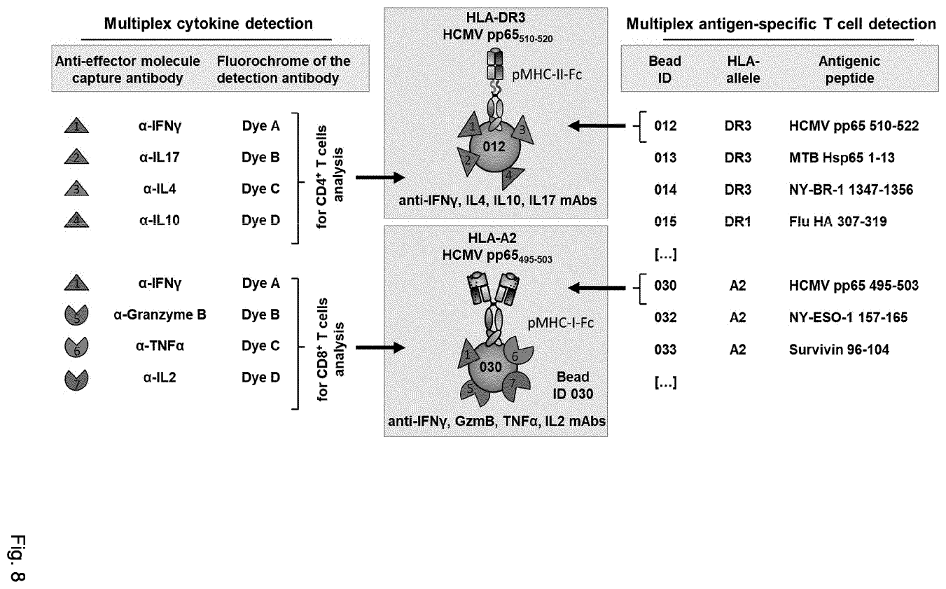

In some versions of these aAPMs a further attachment sequence such as a His₈-tag, a biotinylated AviTag and a cleavage sequence such as a thrombin cleavage site may be present (in amino-to-carboxy terminal order) between the antigen-presenting domain and the dimerization domain such as to provide further versatility of the aAPMs generated. For example, and as shown in FIG. 4, the introduction of such a combination of a further attachment sequence and a cleavage site allows the aAPMs to be used either as dimeric aAPMs shown in FIG. 1 or as aAPMs simply comprising an antigen-presenting domain and an attachment sequence through the separation of the antigen-presenting domain from the dimerization and Ig Fc domains by cleavage such as thrombin cleavage. As such aAPMs generated through cleavage can no longer dimerise, they can serve as monomeric aAPMs on the aAPCs of the present invention. For example, the tetrameric aAPCs shown in FIG. 4 can be assembled using such aAPMs.

Accordingly, the aAPMs used in the aAPCs of the present invention may consist of a single polypeptide sequence comprising in amino-to-carboxy terminal order: an antigen-presenting domain and an attachment sequence, wherein the sequence of the antigen presenting domain comprises an N-terminal antigen peptide sequence.

Notwithstanding, in embodiments where the aAPM comprises an Ig Fc domain, the Ig Fc domain of the aAPM is typically a mouse IgG2a Fc region comprising the constant heavy chain regions 2 and 3 (CH2-CH3) or a human IgG1 Fc (CH2-CH3) region, each containing the aglycan mutation N297Q or N297A. In particular, the IgG Fc region comprises SEQ ID NO:3 or SEQ ID NO:4.

In the aAPMs of the present invention, particularly in the embodiments already described in detail above, the attachment sequence is a peptide tag for attaching the aAPM to a surface, especially for attachment of the aAPM to the surface via affinity-based binding and/or conjugation to the surface.

In some embodiments, the attachment sequence comprises a His-tag (SEQ ID NO 5), a Strep-tag II (SEQ ID NO 6), two Strep-tag II sequences flanking a glycine-serine-rich spacer sequence (SEQ ID NO 7), a Strep-tag II and a C-terminal cysteine residue (SEQ ID NO 8) and/or a biotinylation attachment site (AviTag) (SEQ ID NO 9).

In some embodiments, the above peptide tag sequences can be combined to form an attachment sequence having dual specificity.

The aAPMs of the present invention comprise an antigen peptide sequence. Generally, the antigen peptide sequence is an antigen selected from the group of consisting of: viral antigens; bacterial antigens; fungal antigens; parasite antigens; autoimmune; allergy-related and tumour antigens. Typically, as the aAPMs aim at eliciting an antigen-specific T cell response, for example to identify T cells in a patient sample, which have previously been exposed to the antigen, the antigen peptide sequence are sequences of disease-associated antigens. However, the skilled person will appreciate that the clinical relevance of uncharacterised peptide sequences may also be determined by incorporating them as the antigen peptide sequence of an aAPM of the present invention because, depending on the T cell response elicited and analysed, such peptide antigens can be correlated with a disease phenotype attributed to the patient from whom the T cell sample was obtained.

While a few exemplary antigen peptide sequences are listed below, the skilled person will be aware of the antigen peptide sequences available via the well-established peptide epitope databases accepted in the field such as the Immune Epitope Database (IEDB; accessible online at: http://www.iedb.org or the SYFPEITHI database of MHC ligands database (accessible online at: http://syfpeithi.de/). IEDB lists more than 500,000 peptidic epitopes and SYFPEITHI comprises more than 7000 peptide sequences known to bind class I and class II MHC molecules.

Examples of MHC-I antigen peptide sequences suitable for use with the aAPMs of the present invention are the sequences of SEQ ID NOs 10 to 29.

As already indicated, the above list of potential antigen peptide sequences is by no means exhaustive. In particular embodiments, the antigen peptide sequence is selected from the group consisting of: Human cytomegalovirus pp65 495-503 (SEQ ID NO 10); Epstein-Barr virus BMLF-1 259-267 (SEQ ID NO 13); Influenza virus matrix protein 58-66 (SEQ ID NO 14), NY-ESO-1 157-165/165V (SEQ ID NO 11); and Survivin 96-104/97M (SEQ ID NO 12).

The aAPMs of the present invention, advantageously, can be configured to either comprise an antigen presenting domain suitable to elicit an immune response from CD8⁺ T cells or, alternatively, from CD4⁺ T cells. Accordingly, in some embodiments the antigen-presenting domain of the single polypeptide sequence comprises in amino-to-carboxy terminal order: the antigen peptide sequence, a first linker sequence, and an MHC class I portion. Specifically, in such embodiments the MHC class I portion in amino-to-carboxy terminal order comprises a β₂-microglobulin sequence, a second linker sequence, an MHC class I HLA-A2 α1 sequence, an MHC class I HLA-A2 α2 sequence and an MHC class I HLA-A2 α3 sequence. The first linker sequence suitably comprises a first cysteine residue and the MHC class I HLA-A2 α1 sequence comprises a second cysteine residue, wherein the first and second cysteine residues form a disulfide trap enhancing the association of the antigen peptide sequence to the MHC class I portion of the antigen-presenting domain through covalent linkage. Preferably, the first linker sequence comprises SEQ ID NO 30.

The second cysteine residue of the MHC class I HLA-A2 α1 sequence is the result of a tyrosine to cysteine mutation at position 84 of the MHC class I HLA-A*02:01 α1 sequence. Preferably, the MHC class I portion comprises SEQ ID NO 31.

In addition, in some embodiments the glutamine residue 115 of the MHC class I HLA-A2 α2 sequence is mutated to glutamic acid for enhanced CD8 binding. Preferably, the MHC class I portion comprises SEQ ID NO 32.

In a preferred embodiment, the aAPM comprises in amino-to-carboxy terminal order:

the antigen peptide sequence and SEQ ID NO 33, which includes a β₂-microglobulin sequence (SEQ ID NO 34), a second linker sequence (SEQ ID NO 35), an HLA-A*02:01[Y84C] ectodomain sequence (SEQ ID NO 36), a third linker sequence (SEQ ID NO 37), a mouse IgG2a-Fc[C224S, N297Q] (SEQ ID NO 38 and a Strep-tag II (SEQ ID NO 6) connected to a linker sequence.

In alternative embodiments, the antigen-presenting domain of the single polypeptide sequence comprises in amino-to-carboxy terminal order: the antigen peptide sequence, a first linker sequence, and an MHC class II portion. Specifically, in such embodiments the MHC class II portion in amino-to-carboxy terminal order comprises an MHC class II HLA-DRβ1 sequence and an MHC class II HLA-DRβ2 sequence.

Note that the endoplasmic reticulum (ER) leader sequence of SEQ ID NO 39 (derived/modified from the influenza virus HA1 protein) and SEQ ID NO 40 (derived from the human serum albumin protein) are present in the cDNA generated for MHC class I and MHC class II beta chain aAPMs, respectively, and influence the efficacy of expression/secretion but are cleaved off from the mature protein. This holds true for MHC-I and MHC-II aAPMs.

In some preferred embodiments, DRB1*03:01/DRA-binding peptide ligands can be selected from the peptides of SEQ ID NOs 41 to 46.

The first linker sequence connecting the respective peptide with DRB1*03:01 (or other MHC class II alloforms) ectodomain may comprise the glycine-serine-rich sequence SEQ ID NO 47. CLIP (class II-linked invariant chain peptide, human Invariant Chain 103-117) may be used as universal placeholder peptide such as SEQ ID NO 48 together with a sequence with a thrombin cleavage site connecting CLIP with various MHC class II DRB alloforms such as SEQ ID NO 49.

In some embodiments, the MHC class II portion comprises the DRB1*03:01 ectodomain sequence of SEQ ID NO 50 and/or in amino-to-carboxy terminal order comprises an MHC class II HLA-DRα1 sequence and an MHC class II HLA-DRα2 sequence, wherein the MHC class II portion comprises the DRA*01:01 sequence of SEQ ID NO 51.

Again, the HLA-DRA ER leader sequence of SEQ ID NO 52, which is encoded for in the cDNA of encoding the aAPM, is beneficial for the expression/secretion of the aAPMs as soluble proteins but is cleaved off during processing of the mature protein. As such, the ER leader peptide of the DRA chain (SEQ ID NO 52) is not present in the mature protein of SEQ ID NO 51.

In the aAPMs having an MHC class II antigen presenting domain, the dimerization domain further comprises a parallel coiled-coiled acidic/basic zipper motif, such as a basic zipper motif such as the parallel coiled-coiled basic zipper motif, which comprises SEQ ID NO 53 or, alternatively, an acidic zipper motif, such as the parallel coiled-coiled acidic zipper motif, which comprises SEQ ID NO 54. For example, if SEQ ID NO 53 is present on one chain of the MHC class II heterodimer, then SEQ ID NO 54 is present on the other chain of the MHC class II heterodimer.

Preferably, the aAPM comprises in amino-to-carboxy terminal order: the antigen peptide sequence and SEQ ID NO 55 that is mandatorily co-expressed with SEQ ID NO 56.

As already stated above, the present invention relates to specifically designed aAPMs, which are configured such that they dimerise, either with another aAPM of the same type such as to form a homodimer or, alternatively, with another molecule such as to form a heterodimer in which only aAPM comprises an antigen peptide sequence.

The aAPMs of the present invention are specifically designed such that they dimerise. Accordingly, the present invention also relates to aAPCs comprising a dimer comprising an artificial Antigen Presenting Molecule (aAPM) as described above.

The dimer is either a homo- or heterodimer of two aAPMs comprising MHC class I antigen presenting domains or a heterodimer of an comprising MHC class II antigen presenting domain and a second molecule.

In the MHC class II aAPM heterodimer, the second molecule is a single polypeptide sequence comprising in amino-to-carboxy terminal order: an MHC class II portion corresponding to the MHC class II portion of the aAPM but without an N-terminal antigen peptide sequence, a dimerization domain, which is complimentary to the dimerization domain of the aAPM, an immunoglobulin (Ig) Fc domain and an attachment sequence.

In the heterodimer, the dimerization domain of the second molecule comprises an IgG hinge region complimentary to the IgG hinge region of the aAPM as well as a parallel coiled-coiled acidic/base zipper motif complimentary to the parallel coiled-coiled acidic/base zipper motif of the aAPM.

The attachment sequence of the second molecule of the heterodimer either is the same sequence as the attachment sequence of the aAPM or is a different sequence.

For example, the MHC class II dimer may comprise SEQ ID NO 55 and SEQ ID NO 56 having different attachment sequences.

Exemplary attachment sequences allowing for the attachment of the aAPM are already listed and discussed above. These attachment sequences may also be utilised as the attachment sequences of the second molecule. As such, an attachment sequence such as the Strep-tag can be used to enable binding of the aAPM dimer to beads comprising a specifically engineered streptavidin, i.e. Strep-Tactin. Similarly, a polyhistidine (His-tag) attachment sequence can be used to attach the dimer to nickel nitrilotriacetic acid (Ni-NTA) conjugated beads. Yet further, a site-specific enzymatic biotinylatable tag sequence (i.e. AviTag) can be used to attach the dimer to streptavidin-conjugated beads. Further, attachment to a surface can be mediated by direct conjugation of the aAPM and/or the second molecule N-terminus to the surface of carboxy beads via N-Hydroxysuccinimide)/1-Ethyl-3-(3-dimethylaminopropyl)-carbodiimide (NHS/EDC) crosslinking. Yet further, the IgG2a-Fc domain of can be captured by anti-IgG2a antibody- or Protein A/G-coated beads, thereby attaching the aAPM and/or the second molecule to the surface of the bead. Similarly, when the attachment sequence is a peptide tag, such dimers may be attached to beads coated with antibodies specific for the peptide sequence of the tag such as anti-His-tag or anti-Strep-tag antibodies.

When the attachment sequences are different, the skilled person will be readily equipped to choose suitable variations and combinations of attachment sequences such as combinations of conjugation and affinity-based attachments via peptide tags. Different attachment sequences may be utilized to differentially purify heterodimers (DRA/DRB) and to eliminate possible homodimers (DRA/DRA+DRB/DRB). However, the inventors have identified that the combination of IgG hinge regions and parallel coiled-coiled acidic/basic zipper motifs leads to the predominant formation of the desired heterodimers. In such embodiments, sequential/differential purification may therefore be neglected.

However, when biotinylated constructs are used, such constructs are purified via histidine tags, since Strep-tag-based purification becomes impossible. The Strep-tag II/Strep-Tactin purification system comprises the Strep-tag II sequence (WSHPQFEK), which binds with high selectivity to Strep-Tactin, an engineered/mutated streptavidin. Thus, the Strep-tag allows affinity purification of a protein of interest fused to the Strep-tag via Strep-Tactin resin. Here, desthiobiotin or biotin is used to elute the tagged-protein from the Strep-Tactin, since desthiobiotin and biotin have a higher affinity to StrepTactin as the Strep-tag. Biotin almost binds irreversibly to Strep-Tactin and can only be eluted using NaOH. Consequently, if the protein-of-interest is biotinylated, it will bind irreversible to StrepTactin and can barely be eluted. Thus, another purification system is required.

As such, a peptide tag of the second molecule allows for attachment of the dimer to the surface via affinity-based attachment and/or direct conjugation.

The attachment sequence of the second molecule may be selected form peptide tags comprising SEQ ID NO:5, SEQ ID NO:6, SEQ ID NO:7, SEQ ID NO:8 or SEQ ID NO:9.

FIG. 1 illustrates the domain structure and assembly of an exemplary MHC-I dimer (aAPM) for use in the aAPCs of the present invention: The exemplary disulfide-trapped (dt) peptide-MHC-Class I (pMHC-I) immunoglobulin Fc (Fc) aAPM dimer shown comprises two single polypeptide chains comprising a covalently linked T cell epitope peptide ligand (8-11 amino acids), human β2-microglobulin (β2m), HLA-class I allele ectodomain and constant heavy chain (CH) domains 2 and 3 of murine immunoglobulin isotype IgG2a. Dotted lines indicate flexible glycine-serine linkers. The intramolecular disulfide trap between the C-terminal peptide extension and a cysteine (C) residue replacing MHC-I tyrosine (Y) 84 residue provides further stabilization of the pMHC complex. The C-terminal Strep-Tag II (STag) sequence allows affinity-purification under neutral conditions.

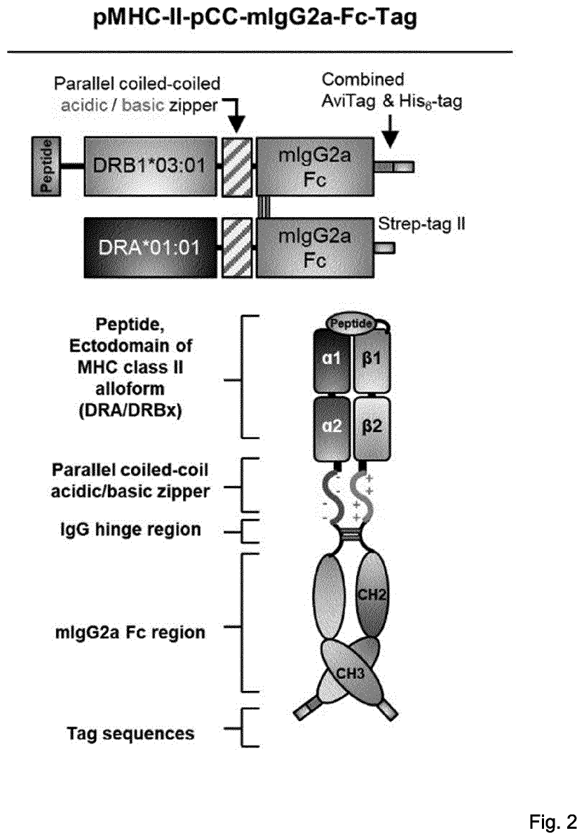

FIG. 2 illustrates the domain structure and assembly of an exemplary MHC-II dimer (aAPM) for use in the aAPCs of the present invention: The exemplary peptide-MHC-Class II (pMHC-II) monomer immunoglobulin Fc fusion aAPM dimer shown comprises an MHC-II aAPM polypeptide chain and a second polypeptide chain (second molecule). The MHC-II aAPM polypeptide chain is an MHC-II β-chain, which is N-terminally fused with an antigenic peptide via a flexible glycine-serine linker. The C-terminus of β-chain ectodomain (31-β₂) is fused to a parallel coiled-coil (pCC) basic zipper followed by the hinge domain and CH2 and CH3 of mlgG2a (Fc) and a C-terminal hexahistidine (Hiss)-tag and AviTag for site-specific enzymatic biotinylation. The second molecule is a polypeptide chain comprising an ectodomain of the monomorphic MHC-II α-chain (α1-α2), which is C-terminally fused to a complementary acidic pCC-Fc and a C-terminal Strep-Tag II.

In a first aspect, the present invention relates to an artificial antigen-presenting cell (aAPC) for the detection of effector molecules of a T cell in response to presentation of an antigen peptide sequence, the aAPC comprising:
(a) a surface, wherein the surface is the surface of a particle, optionally of a bead;
(b) one or more artificial Antigen Presenting Molecules (aAPMs), wherein the aAPMs are attached to the surface via their attachment sequences or via direct chemical conjugation; and
(c) one or more capture molecules attached to the surface,

wherein each of the one or more aAPMs and/or each of the one or more dimers comprises an identical antigen peptide sequence.

Particles or beads ranging between 0.5 to 50 μm in diameter, in particular between 0.5 to 40 μm, in particular between 0.5 to 30 μm, in particular between 0.5 to 20 μm, in particular between 0.5 to 10 μm, in particular between 2.5 to 7.5 μm, in particular between 3 to 7 μm, in particular between 4 to 7 μm, in particular between 5 to 7 μm, such as 6.5 μm, are suitable as particles or beads to which the aAPMs may be attached. In a particular embodiment of the aAPC of the present invention, the particles are magnetic MagPlex® microspheres developed and provided by the Luminex Corporation having a diameter of approximately 6.5 μm (hereinafter referred to as “Luminex beads”).

The surface of particles or beads to which the aAPMs are to be attached to form an aAPC of the present invention must be suitable for attachment of the aAPMs described above via the aAPMs respective attachment sequences. In particular, the particles or beads can comprise moieties on their surface compatible with the corresponding moieties of the attachment sequences of the aAPMs. For example, if:
(a) the attachment sequence of the aAPM comprises a biotin sequence, the particle or bead is streptavidin-coated;
(b) the attachment sequence of the aAPM comprises a Strep-tag sequence, the particle or bead is Strep-Tactin-coated;
(c) the attachment sequence of the aAPM comprises an Ig Fc sequence, the particle or bead is coated with anti-Fc antibodies or protein NG; and so forth.

The capture molecules of the aAPC allows for the capture of effector molecules released by the T cells in response to antigen presentation by the aAPC. Depending on the aAPMs, particularly depending on the combination of MHC class I and/or class II aAPMs, of the aAPC the effector molecules may be released by CD4⁺ and/or CD8⁺ cells. As such, the one or more capture molecules are one or more capture antibodies specific for one or more effector molecules released from, optionally secreted from, a T cell in response to presentation of the antigen peptide sequence. For example, the one or more capture antibodies may be specific for the same effector molecule or may comprise one or more groups of capture antibodies, each group being specific for a different effector molecule. The one or more capture molecules may be capture antibodies specific for one or more effector molecules selected from the group consisting of: Interferon gamma (IFN-γ); interleukin-2 (IL-2); IL-4; IL-5; IL-6; IL-9; IL-10; IL-13; IL-17; IL-21; IL-35; granzyme B; tumour necrosis factor alpha (TNF-α); lymphotoxin alpha (LT-α), and transforming growth factor beta (TGF-⊖). This list is exemplary in nature and not to be considered exhaustive. Notwithstanding, as will be evident to the skilled person, the quality/affinity of a capture antibody has a significant effect on efficiently capturing effector molecules.

Enhancement of a T cell response to a presented antigen can be achieved by exposing the T cell to co-stimulatory molecules when antigen presentation occurs. Many such co-stimulatory molecules have been described and some are particularly useful in the present invention. Exposure of the T cell to co-stimulatory molecules for enhancing the T cell's response to presentation of the antigen peptide sequence can either be achieved: (a) by using soluble co-stimulatory molecules; or (b) by using aAPCs of the invention, which further comprise one or more immobilized co-stimulatory molecules attached to their surface.

Option (a) may be preferable compared to option (b) in some instances as it allows for maximal sensitivity of the aAPC and therefore of the assays in which they are used, because the capacity of the aAPC surface for capture molecules is not reduced due to the attentional attachment of co-stimulatory molecules. Notwithstanding, when pursuing option (b) co-stimulatory molecules may be attached to the aAPC by similar means than the aAPMs themselves. For example, the co-stimulatory molecules can be fusion proteins comprising an N-terminal stimulatory domain and an immunoglobulin (Ig) Fc domain and, optionally a further C-terminal attachment sequence. In particular, the co-stimulatory molecules are suitable for attachment to the aAPCs by affinity-based attachment and/or direct conjugation. Exemplary co-stimulatory molecules are fusions of the stimulator domains of ICAM1 (CD54) or LFA-3 (CD58) and the mlgG2a-Fc portion.

When exposing a pure and pre-characterised T cell population, i.e. a T-cell line, which has only one antigen specificity and is “bystander T cell-free”, to aAPCs of the invention in combination with specific co-stimulatory molecules previously described as enhancing the T cell activation, the inventors found no such additionally enhanced T cell activation indicating the highly efficient stimulation of the T cells by the aAPCs of the invention even in the absence of co-stimulatory molecules. In particular, it was noted that anti-CD28/anti-4-1BB or anti-CD2 antibodies did not improve the T cell responses determinable in T cell response assays utilising the aAPCs of the present invention. For example the inventors used pure CD8⁺ T cell lines derived from various healthy donors that were specific for the human cytomegalovirus (HCMV) pp65₄₉₅₋₅₀₃/HLA-A*02:01 pMHC-I complex. Those virus-specific T-cells were: (1) identified in HLA-A*02:01 allele expressing (HLA-A2⁺) healthy donor derived peripheral blood mononuclear cells (PBMCs) using a pMHC multimer staining; (2) expanded using the cognate peptide; (3) isolated using a pMHC-Fc (aAPM described by this invention) and magnetic beads; and (4) again expanded using irradiated allogenic HLA-A2⁺ PBMC (feeder cells) loaded with the cognate peptide to obtain a pure T cell line.

The effects of co-stimulatory molecules on T cell samples obtained from tumour patients is, however, still considered to be beneficial for increasing the sensitivity of such assays. In particular, as the assays employing the aAPCs of the invention are particularly suitable to determine patient-specific T cell responses to tumour-specific antigens, i.e. to allow the immunological characterisation of a patient's T cell population and therefore to allow for the characterisation of the patient's tumour's antigenic makeup.

Moreover, also recombinant human 4-1BBL (CD137L), human CD70, human B7-1 (CD80) or B7-2 (CD86) can serve as stimulatory domains in co-stimulatory molecules suitable for use with the aAPCs of the present invention, in particular if they are fused to an Ig Fc. Such recombinantly produced co-stimulatory Ig Fc fusion molecules may have increased co-stimulatory effects on T cells compared the corresponding anti-CD28 or anti-4-1BB antibodies binding to the same co-stimulatory receptor. In some embodiments, the co-stimulatory molecules may be attached to the same aAPCs as the aAPMs. In others, they may be attached to different substrates to be made available to the T cells at the same time or at least in such close temporal sequence to exposure of the T cells to the aAPC that their stimulatory effect on the T cell is still ongoing when the T cell is presented with antigen by the aAPC. In some embodiments, the co-stimulatory molecules may not be attached to a surface or substrate but may simply be available for T cell stimulation as soluble components of the T cell culture medium.

The one or more co-stimulatory molecules are one or more co-stimulatory antibodies, optionally selected from antibodies specific for one or more cell surface-expressed co-stimulatory T cell receptor selected from the group consisting of anti-CD2, anti-CD28, anti-CD27, anti-CD134, anti-CD137, or recombinant costimulatory molecules such as such as B7-1 (CD80), B7-2 (CD86), ICAM-1 (CD54), LFA-3 (CD58), 4-1BBL (CD137L) and OX40L (CD252), CD40L (CD154) and CD70.

In one or more preferred embodiment the one or more co-stimulatory antibodies are specific for the same co-stimulatory receptor or the one or more co-stimulatory antibodies comprise one or more groups of co-stimulatory antibodies, each group being specific for a different group of the same co-stimulatory receptors.

The aAPCs of the present application provide for various levels and/or degrees of multiplexation. For example, the particles of the aAPCs may be coded to be identifiable and separable from other particles, optionally the particle is colour-coded. In such embodiments, the colour code is indicative of the antigen peptide sequence of the aAPM attached to the aAPC. As such, colour-coded particles can be separated by flow cytometric analysis. In addition or alternatively, the particle may be magnetic. Sorting and separating the aAPCs based on the specific antigen or combination of antigens presented to the T cells allows for the analysis of effector molecules captured by the capture molecules of the aAPC. In light of the interaction with an individual T cell with an individual aAPC and the resulting physical proximity of the T cell and the aAPC allows effector molecules released by the T cell to be captured by the aAPC presenting a specific antigen to the cell. As such, the ability to separate aAPCs based on the identity of the antigen they present and/or the capture molecule attached to the aAPC allows for the dissection of the combination (profile) of effector molecules released from T cells in response to a particular antigen.

For the use in the assays of the present invention, a composition comprising a plurality of aAPCs according to the first aspect is disclosed as a second aspect of the invention, wherein the composition comprises a plurality of identical aAPCs. The identical aAPCs comprise a single capture molecule specific for a respective single effector molecule or the identical aAPCs comprise several capture molecules specific for several respective effector molecules. Such compositions are particularly useful for assessing the profile of effector molecules released in response to presentation of a specific antigen in an assay of a third aspect of the present invention described further below.

Alternatively, in compositions of the second aspect, all aAPCs of each group are coded identically and the antigen peptide sequence presented by the aAPCs of each group is identical within the group but different between each of the groups. Preferably, the aAPCs of each group comprise a single capture molecule specific for a respective single effector molecule, or the aAPCs of each group comprise several capture molecules specific for several respective effector molecules.

Such compositions provide for further degrees and/or levels of multiplexation. In particular, they are useful for assessing several T cell responses in a population of T cells to different aAPCs, i.e. to different antigens being presented, as well as for detecting a plurality of effector molecules by including a plurality of capture antibodies on the aAPCs in an assay of fourth aspect of the present invention described further below.

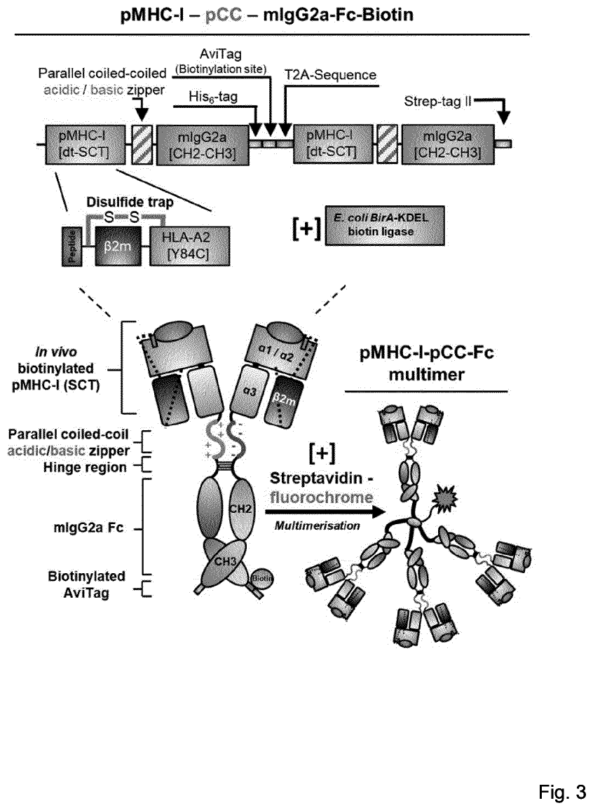

FIG. 3 illustrates the domain structure of an exemplary polycistronic MHC-I aAPM construct and its assembly into an MHC-I aAPM for use in the aAPCs of the present invention: The exemplary polycistronic MHC-I aAPM construct shown is a pMHC-I-heterodimeric biotin-tagged Fc construct (pMHC-I-pCC-Fc) consisting of two separate polypeptide chains co-expressed in a single vector via a T2A sequence. The first aAPM polypeptide chain comprises the pMHC-I portion as single-chain-trimer (SCT) (disulfide-trapped peptide ligand, β2m, HLA-class I allelic ectodomain) fused to a parallel coiled-coil (pCC) basic zipper followed by the hinge domain and CH2 and CH3 of mlgG2a (Fc) and a combinatorial C-terminal attachment sequence comprising a Hiss-tag and AviTag for site-specific biotinylation. The second aAPM polypeptide chain comprises the same pMHC-I portion but comprising complimentary acidic pCC and Fc domains as well as a C-terminal Strep-Tag II attachment sequence. The pMHC-I-pCC-Fc aAPM shown is site-specifically biotinylated in vivo by means of co-expression of BirA ligase molecules fused to the ER retention signal sequence KDEL (BirA-KDEL). The resulting biotinylated pMHC-I-pCC-Fc-Bio aAPM can be multimerized to pMHC-I octamers on a streptavidin surface or can be immobilized on the surface of a streptavidin-coated bead to generate an aAPC of the invention as shown.

FIG. 4 illustrates the domain structure of a further MHC-I aAPM construct and its assembly into an MHC-I aAPM for use in the aAPCs of the present invention: The exemplary pMHC-I-homodimeric biotin-tagged aAPM construct has a cleavable Fc region (pMHC-I-AviTag-TCS-Fc). In particular, the construct shown comprises a single polypeptide chain, which in turn comprises the pMHC-I complex as single-chain-timer (SCT, as described for FIG. 3 above) fused to an octahistidine (His₈)-tag and AviTag followed by a thrombin-cleavage site (TCS) sequence. The cleavage site is C-terminally fused to the hinge domain and CH2 and CH3 of mlgG2a (Fc) and an optional Strep-Tag II (C-terminal of Fc). The pMHC-I-AviTag-TCS-Fc aAPM construct is site-specifically biotinylated in vivo through the co-expression of BirA ligase fused to the ER retention signal sequence KDEL (BirA-KDEL). To generate biotinylated pMHC-I aAPM monomers, the Fc portion is cleaved off by thrombin. pMHC-I-biotin aAPM monomers can be multimerized to pMHC-I multimers using soluble streptavidin or can be immobilized on the surface of a streptavidin-coated bead to generate an aAPC of the invention as shown.

In the third aspect, the present invention relates to an assay for determining an antigen-specific T cell response, the assay comprising the following steps:
(a) contacting T cells with a composition according to the second aspect under conditions and for a time suitable to elicit a response from the T cells upon presentation of the antigen peptide sequence by the identical aAPCs;
(b) separating the aAPCs from the T cells;
(c) contacting the aAPCs with detection antibodies against one or more effector molecules for which the one or more capture molecules of the aAPCs are specific;
(d) analysing the release of effector molecules of the T cells by detecting the effector molecules captured by the one or more capture molecules of the aAPCs, thereby determining the T cell response specific to the antigen peptide sequence presented by the identical aAPCs.

In the fourth aspect, the present invention relates to an assay for determining a plurality of antigen-specific T cell responses, the assay comprising the following steps:
(a) contacting T cells with a composition according to the second aspect under conditions and for a time suitable to elicit responses from the T cells upon presentation of each of the different antigen peptide sequences presented by each group of aAPCs;
(b) separating the aAPCs from the T cells;
(c) contacting the aAPCs with detection antibodies against one or more effector molecules for which the one or more capture molecules of the aAPCs within each group of aAPCs are specific;
(d) identifying and separating the aAPCs of each of the groups of aAPCs;
(e) analysing the release of effector molecules of the T cells by detecting the effector molecules captured by the one or more capture molecules of the aAPCs within each group of aAPCs, thereby determining a plurality of antigen-specific T cell responses specific to each antigen peptide sequence presented by each group of aAPCs.

Regarding the conditions and time suitable to elicit responses from the T cells in the assays for the third and fourth aspect of the invention certain key parameters have been identified as also illustrated by the Examples below. However, the skilled person may determine the most suitable conditions for successful performance based on his or her common general knowledge in relation to the culture conditions and parameters necessary to ensure adequate maintenance of T cells being used in the T-Plex Assay as well as by employing routine experimentation.

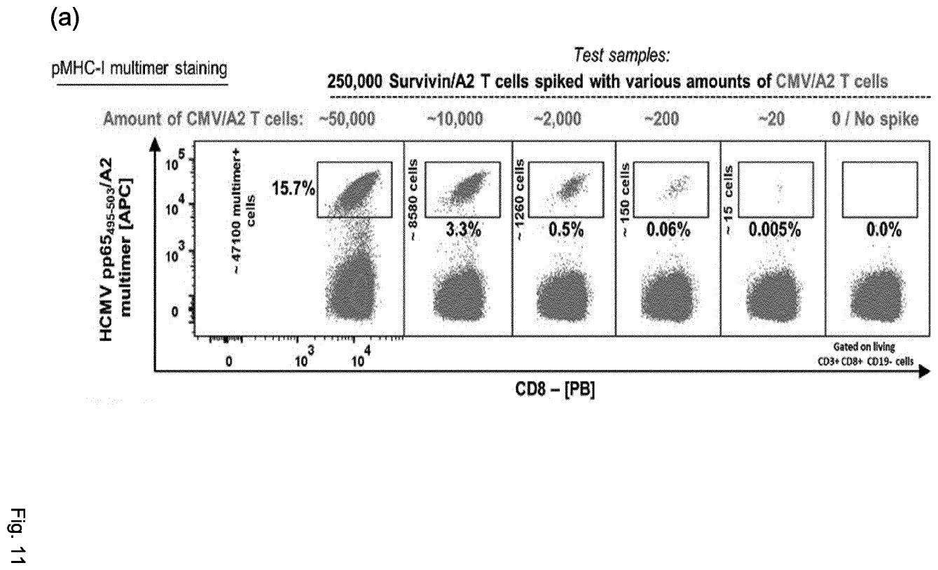

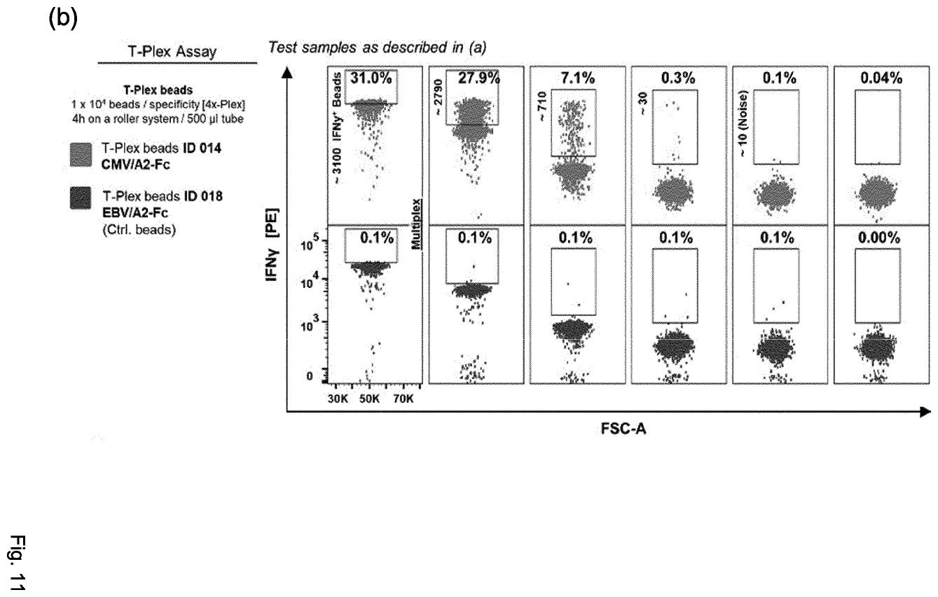

For successful assay performance, it is important that during the step of contacting T cells with a composition comprising the aAPCs, the sample is kept in motion such as to generate an evenly mixed suspension of aAPCs and T cells. Such motion can, for example be generated by gently but constantly rolling the reaction vessel on a laboratory roller (“One-tube reaction”) as also illustrated in FIG. 6(b). For example, an assay tube (diameter ˜1 cm) is placed horizontally between the gap of two rollers (diameter ˜3 cm) of the mixing/rolling device leading to rotation of the assay tube along its longitudinal axis. A given number of rounds per minute (RPM) of the device leads to an approximately duplicated RPM of the assay tube, thereby allowing for suitable reaction conditions while minimizing any risk of sample cross-contamination.

In particular, and without being bound by theory, it would appear that the constant movement limits cross-contamination (or “cross-bleeding”) of soluble effector cytokines i.e. IFN-γ on bystander aAPCs that are linked to a non-matching aAPM, which, under static conditions, would be in close proximity to an activated T cell that is contacting an aAPC with a matching aAPM. In other words, constantly rolling the reaction vessel can prevent cross-contamination/cross-bleeding of effector molecules captured on aAPCs that have not elicited a T cell response. Alternatively, for non-rollable reaction vessels, such as 12-, 24-, 48-, and 96-well plates, the test sample may be kept in motion by way of 3D/orbital movement of the vessels such as illustrated in FIG. 6(a).

Alternatively, the different bead colours can also be spatially separated and immobilized within a shared assay room (spotted beads on a 6-well, which has a magnet below), followed by orbital/3D mixing movement for the test sample. Further, when employing rollable reaction vessels, it has proven beneficial with respect to achieving an increased assay sensitivity to compact the cells and the aAPCs by centrifugation just prior to the rolling. Without wanting to be bound by theory, the beneficial effect of this initial centrifugation most likely enhances the chance that the aAPCs and cognate T cells are placed in close proximity within the pellet, i.e. within close proximity of each other right from the start of the assay due to the aAPCs and

T cells having similar physical characteristics such as shape (diameter) and weight. For example, a human T cell has a diameter in the range of 7-9 μm, while the Luminex beads used in some embodiments of the aAPCs of the invention have a diameter of approximately 6.5 μm. As such, the T cells and aAPCs are likely to be present in similar volumes of the three-dimensional space of the reaction volume, i.e. are within close proximity of each other within the pellet. This likely increases the chances that the aAPCs and T cells interact with each other directly.

In addition, the sensitivity of the assay is dependent on the specificity and affinity of the capture molecules used on the aAPCs. In some embodiments the sensitivity of the assay could be improved by about 2-fold by using a “better” anti-IFN-γ antibody clone as the capture molecule. Moreover, 30-50% of the aAPCs' bound molecules need to be pMHC (an APM) to trigger a proper T cell response when contacting the T cells with a composition comprising aAPCs under suitable conditions for about 4 to 6 h in case IFN-γ, IL-2 and TN F-α are intended effector molecules to be captured.

The time period of 4 to 6 hours seems to be optimal and in line with the observations reported in the literature where it was shown that the secretion of IFN-γ by activated CD8⁺ T cells has a lag phase of roughly 90 minutes (Dushek et al, Sci. Signal. 4(176):ra39, 2011) and that IFN-γ secretion peaks after 4 h of initial stimulation of T cells (Betts et al, J. Immunol. Methods. 281(1-2):65-78, 2003), whereas longer exposure times increase background signals caused by bystander effector molecule capture on aAPCs that have not elicited a T cell response.

In the assays of the third and fourth aspect, the T cells are purified T cells.

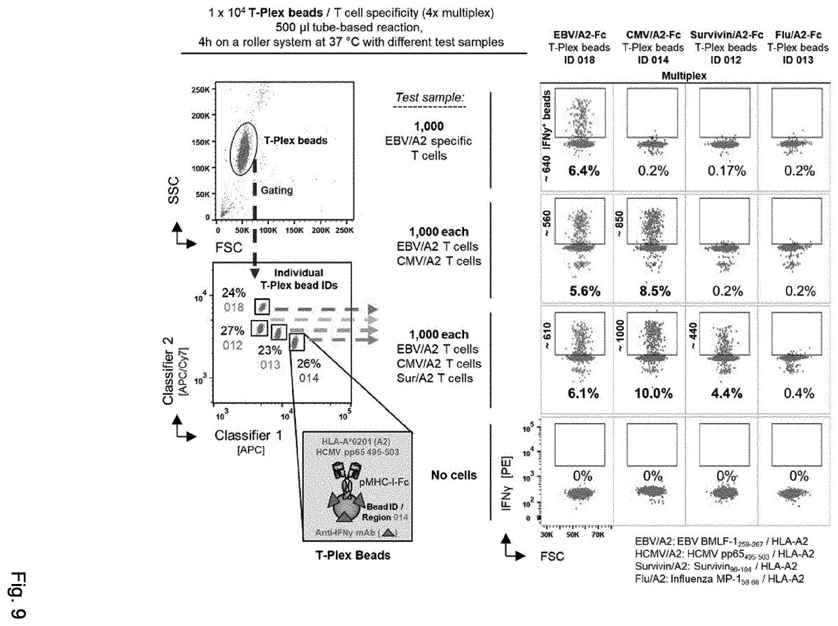

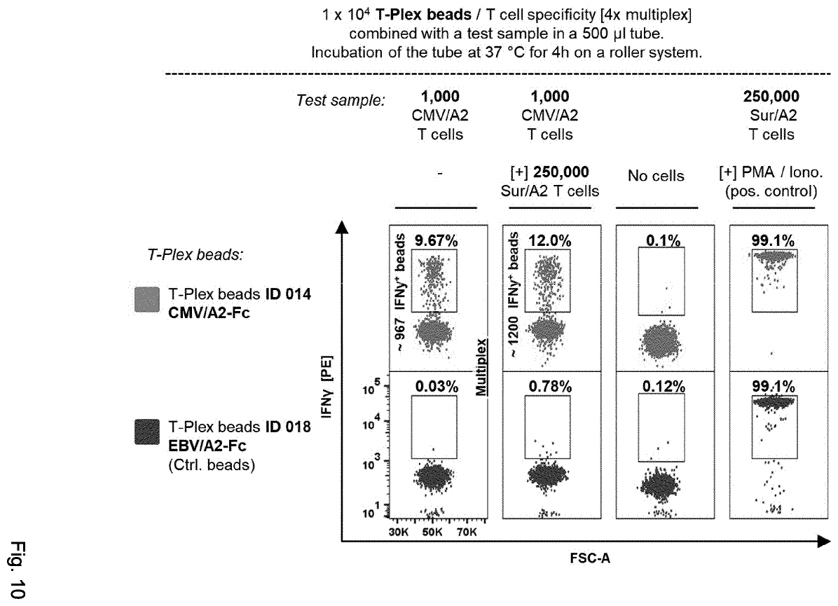

FIG. 5 illustrates the assembly of an aAPC of the present invention for use in a multiplex assay for the determination of an antigen-specific T cell response in accordance with the assays and methods described. In particular, the use of aAPCs of the present invention for linking T cell specificities to defined bead colours is shown to illustrate a first level of multiplexation made possible through the aAPCs of the present invention. (a) aAPC concept: The exemplary aAPCs for use in the assays of the present invention shown are bead-based colour-coded aAPCs with T cell effector molecule capture capacity, in particular with the capacity to capture the cytokine interferon-γ (IFN-γ). The aAPCs are assembled by coupling defined pMHC-I or pMHC-II and other optional co-stimulatory molecules together with an effector cytokine-capture antibody to a colour-coded bead. (b) Architecture and assembly of an exemplary aAPC of the present invention: In a first step, IFN-γ capture antibodies (murine IgG1 isotype) and monoclonal rat anti-murine IgG2a antibodies are covalently conjugated in a 3 to 2 ratio to coloured carboxylated magnetic polystyrene microparticles (MagPlex® Microspheres from Luminex Corp., hereinafter referred to as “Luminex beads”). In a second step, the so-prepared beads are loaded with saturating amounts of pMHC-1-mIgG2a-Fc aAPMs by using crude supernatants from aAPM-expressing CHO-S cells or, alternatively, by using affinity-chromatography purified pMHC-1-mIgG2a-Fc. (c) aAPC co-coordinate system: Shown are 30 different aAPC pools using the above-described Luminex bead based assemblies and their respective regions (position) in a two-dimensional dot plot as measured by a flow cytometer. Each aAPC pool can be easily linked to defined T cell epitope through conjugation with respective pMHC-I or pMHC-II aAPMs. (d) Basic principle of a multiplex assay: pMHC-Fc and anti(α)-IFN-γ capture antibody coupled colour-coded aAPCs (T-Plex beads) activate cognate T cells in an antigen-specific manner, which drives IFN-γ secretion of that activated T cell. The secreted IFN-γ is proximally captured on the same bead and can be detected by a fluorochrome-labeled αIFN-γ detection antibody. T-Plex beads can be subsequently analysed in a suitable bead-analyser instrument (FACS) based on their intrinsic colour (bead classifier) and their IFN-γ load. dt-pMHC-I-Fc dimer: Disulfide-trapped (dt) peptide-MHC-Class I (pMHC-I) immunoglobulin Fc (Fc) dimers; mAb: monoclonal antibody.

FIG. 6 illustrates two differing exemplary T-Plex Assay workflows. (a) T-Plex Assay workflow based on bead spotting followed by orbital shaking: To enable a multiplex assay and avoid IFN-γ cross-bleeding, colour-coded T-Plex beads loaded with different pMHC-Fc aAPMs are spotted onto individual magnetic reaction fields generated by means of a 96-well magnet placed below a suitable reaction chamber (6-well plate or lid of plate). T cells are added to the medium-flooded chamber and the whole reaction is incubated for 4 to 6 hours at 37° C. under gentle and constant 3D orbital agitation. In a final step T cell and beads can be magnetically separated from T cells. All beads are then pooled and their IFN-γ load is analysed. (b) T-Plex “rotation-one-tube-reaction” principle: Colour-coded T-Plex beads loaded with different pMHC-Fc aAPMs are filled into a conically skirted tube combined with CO₂-satured assay medium and the T cell sample to be analysed. The assay tube is closed and rotated for 4 to 6 h at 37° C. as shown. Finally, magnetic beads are collected by means of a magnet and washed. Subsequently T-Plex beads are analysed for their IFN-γ load.

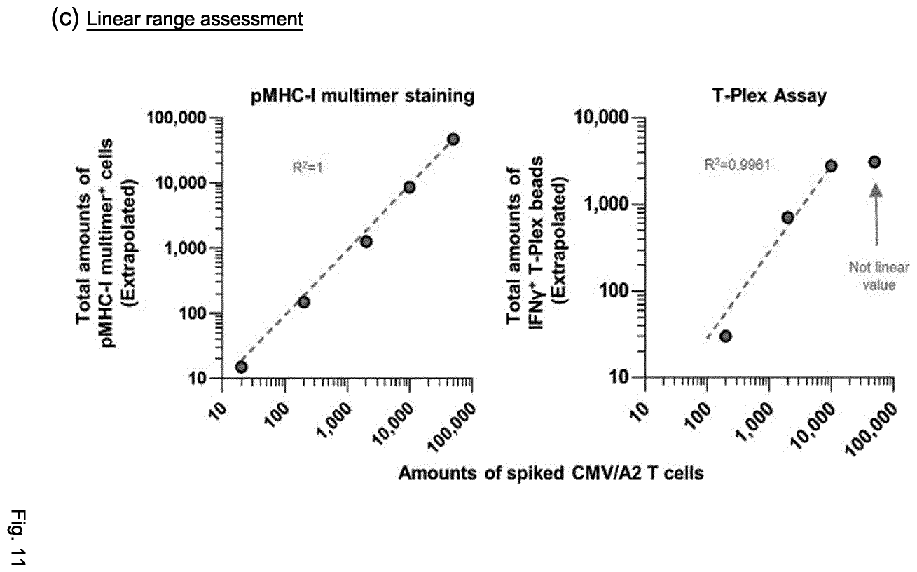

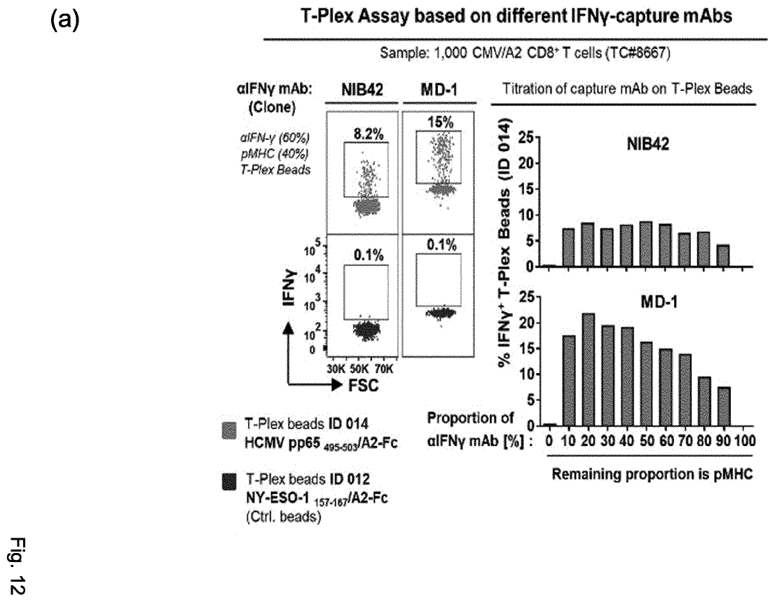

FIG. 7 illustrates the assembly of a further aAPC of the present invention for use in a multiplex assay for the determination of an antigen-specific T cell response in accordance with the assays and methods described. In particular, the use of aAPCs of the present invention for antigen-specific CD4⁺ T cell detection and parallel functional profiling is shown to illustrate several levels of multiplexation made possible through the aAPCs of the present invention: Multiplex-based antigen-specific CD4⁺ T cell detection and parallel functional profiling. Two differing exemplary T-Plex Assay workflows are illustrated. (a) For multiplex-based antigen-specific and functional phenotype profiling of CD4⁺ T cell populations, aAPCs comprising a variety of different capture antibodies (T-Plex² beads) are assembled by coupling a specific aAPM such as an pMHC-II-pCC-Fc and other optional co-stimulatory molecules together with several different effector cytokine-capture antibodies to colour-coded beads (T-Plex² beads). (b) The T-Plex² beads (aAPCs) activate cognate CD4⁺ T cells in an antigen-specific manner, which drives secretion of cytokines depending on the functional phenotype and of the activated T cell. A defining cytokine for a Th1 CD4⁺ T cells is IFN-γ, whereas Th2 differentiation rather leads to secretion of IL4, Th17 differentiation to IL17 and Treg differentiation to IL10. The secreted cytokines are captured proximally on the same bead and can be detected by a detection antibody panel labelled with different fluorochromes (dye A-D) depending on the cytokine.

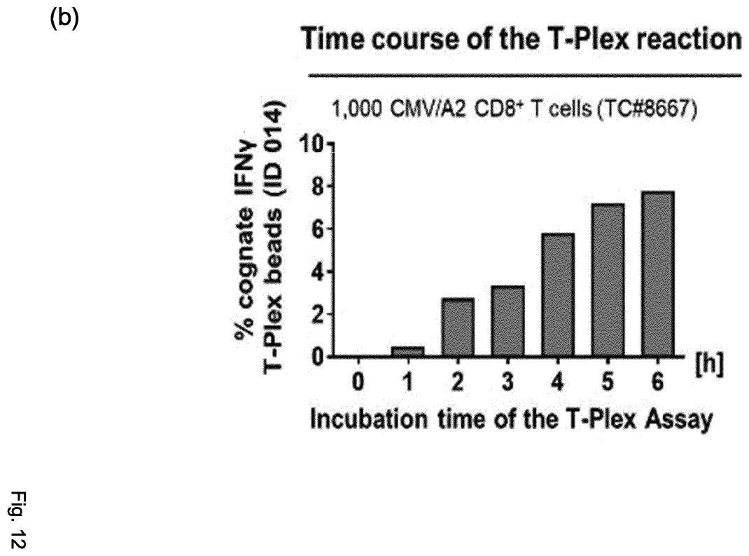

FIG. 8 illustrates exemplary assembly concepts for the design of CD4⁺ and/or CD8⁺ T-Plex² assays in accordance with the assays and methods described: Conjugation of different cytokine-capture antibodies combined with cytokine detection antibodies conjugated to different fluorochromes allows two-level multiplexation, i.e. detection in two dimensions (T-Plex²). The first dimension of the multiplex assay is encoded by the internal colour aAPC and reflects the T cell antigen specificity, whereas the second dimension is based on detecting multiple different cytokines on the same T-Plex bead during cognate T cell stimulation. This allows for the differentiation and analysis of functional profiles of the antigen-specific CD4⁺ T cell as well as CD8⁺ T cell pools within a single assay reaction.

FIG. 9 illustrates the T-Plex Assay proof-of-concept. In particular, the results of the experiment described in Example 1 below highlight the multiplex detection capacity of the T-Plex Assay using three different pure antigen-specific CD8⁺ T cell lines (model system)/pMHC-I.

In particular, Examples 1 to 7 illustrate assays according to the third and fourth aspect of the invention. The assays of the third and fourth aspect may comprise the additional step of separating aAPCs from the T cells comprises washing the aAPCs under conditions suitable to maintain viability of the T cells and subsequently collecting the separated T cells for further in vitro cell culture. Such T cell collections are described in detail in Example 8 below.

In a fifth aspect, the present invention relates to a vector comprising
(a) a polynucleotide sequence encoding the single polypeptide sequence of the aAPM as defined for the first aspect, or
(b) a polynucleotide sequence for polycistronic expression of both peptide chains of a dimer as defined for the first aspect.

Sequence information regarding the polynucleotide vector sequences suitable for encoding the single polypeptide sequence of the aAPM of the first aspect of the invention as well as for polycistronic expression of both peptide chains of a dimer of the second aspect are provided as SEQ ID NO 57 and 58, respectively.

In yet a further aspect, the present invention also relates to a method of manufacturing an aAPC according to the first aspect, wherein the method comprises covalently attaching
(a) an aAPM, preferably an aAPM is a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigen peptide sequence, and
(b) a capture molecule, preferably a capture antibody, to the surface of a microsphere, preferably a colour-coded microsphere.

### EXAMPLES

The invention is further described by the following non-limiting Examples.

### Example 1: T-Plex Assay-Based Antigen-Specific Multiplex Detection of a Defined Set of T Cell Lines

This experiment highlights the multiplex detection capacity of the T-Plex Assay using three different pure antigen-specific CD8+ T cell lines (model system)/pMHC-I.

Four different colours (Luminex bead ID/region: 012, 013, 014, 018) of precursor T-Plex beads (anti-INFγ mAb and anti-mlgG2a-Fc (Fc) mAb conjugated Luminex beads) were loaded with a defined set of pHLA-A2-Fc aAPMs. 10,000 beads per aAPM-loaded T-Plex bead ID/T cell epitope were combined with the indicated amounts of antigen-specific T cell lines in one cone-shaped 500 μl tube filled with ˜500 μl 002-saturated cell medium. The T-Plex Assay was performed at 37° C., 4 h, 40 rpm. The presence of a T cell line was indicated by appearance of an IFN-γ-loaded (IFN-γ⁺ beads) subpopulation of cognate T-Plex beads that was above control beads as shown in FIG. 9. EBV BMLF-1₂₅₉₋₂₆₁/HLA-A2 (A2) specific CD8⁺ T cell line #0144 (EBV/A2 T cells), HCMV pp65₄₉₅₋₅₀₃/A2 specific CD8⁺ T cell line #416 (CMV/A2 T cells) and Survivin₉₆₋₁₀₆/A2 specific CD8⁺ T cell line (Sur/A2 T cells) were used. The rows of T-Plex data in FIG. 9 represent analyses from the same reaction/bead mix (multiplex detection). Used pHLA-A2-Fc aAPMs for T-Plex bead assembly: Survivin₉₆₋₁₀₄/HLA-A2-Fc (Survivin/A2-Fc), Influenza MP-1₅₈₋₆₆/A2-Fc (Flu/A2-Fc), HCMV pp65₄₉₅₋₅₀₃/A2-Fc (CMV/A2-Fc) and EBV BMLF-1₂₅₉₋₂₆₇/A2-Fc (EBV/A2-Fc).

### Example 2: Bystander T Cells do not Decrease the Sensitivity of the T-Plex Assay

Here, a to-be-detected T cell line was embedded (spiked) into bystander (ctrl) T cells in order to analyse whether a certain amount of bystander T cells would spoil the detection sensitivity of the T-Plex Assay. However, as shown by the results illustrated in FIG. 10, this what not the case.

pMHC-I-Fc aAPM loaded aAPCs (i.e. 10,000 beads per T-Plex bead ID/T cell epitope) were combined either with 1,000 HCMV pp65₄₉₅₋₅₀₃/A2-specific CD8⁺ T cell line #416 (CMV/A2 T cells) or 1,000 CMV/A2 T cells spiked into 300,000 bystander Survivin₉₆₋₁₀₆/A2-specific CD8⁺ T cells (Sur/A2 T cells). The T-Plex Assay was performed in a 500 μl tube rotating at 37° C. for 4 h at 40 rpm. The presence of a T cell line was indicated by appearance of an IFN-γ⁺ subpopulation of cognate T-Plex beads in FIG. 10. Functionality of Sur/A2 T cells and all used T-Plex beads was verified by phorbol 12-myristate 13-acetate (PMA)/ionomycin (iono.) based stimulation. In this experiment four different colours (Bead ID: 012, 013, 014, 018) of T-Plex beads were loaded with a defined set of pHLA-A2-Fc aAPMs. In FIG. 10, only the IFN-γ signal of the cognate CMV/A2 T-Plex beads ID 14 (in blue/light grey) and one representative corresponding ctrl-T-Plex bead signal ID 018 (in dark grey) is shown. Pairs of upper and lower FACS-plots represent data analysis from the same reaction/bead mix (Multiplex detection). In this experiment pHLA-A2-Fc aAPM NY-ESO-1₁₅₇₋₁₆₅/HLA-A2-Fc was used instead of Survivin₉₆₋₁₀₄/HLA-A2-Fc for T-Plex bead assembly.

### Example 3: pMHC-I Multimer Staining in Comparison to T-Plex Assay of T Cell Line Spiked Samples

A comparison of one “gold-standard” assay (pMHC-I multimer staining) with a T-Plex Assay according to the present invention using two T cell lines (model system) was performed and the results are illustrated in FIG. 11.

pMHC multimer staining refers to the flow cytometry-based detection of antigen-specific T cell receptors expressed on the T cell surface using soluble MHC-peptide oligomers (multimers), such as purposely designed MHC dimers, pentamers and/or higher order oligomers, as well as biotin-streptavidin-based tetramers that are covalently linked to a fluorochrome (Altman et al., Science 274(5284): 94-96, 1996). Using this method respective antigen-specific T cells are directly visualized upon binding of a matching/cognate pMHC multimer to the T cell receptor expressed on the cell surface of that particular cell.

A defined amount (˜40-100,000) of HCMV pp65₄₉₅₋₅₀₃/A2 specific CD8⁺ T cell line #416 (CMV/A2 T cells) were spiked individually into a pool of 500,000 Survivin₉₆₋₁₀₆/A2 specific T cells. The spiked test sample was split in half and either analysed by commercial pMHC-I multimer staining (a) or T-Plex Assay (b). FIG. 11(a) pMHC-I multimer stainings: The spiked sample was stained with a commercial CMV/A2 pMHC-I pentamer (from ProImmune) in the presence of 50 nM dasatinib. The frequency of pMHC-multimer⁺ within the CD8⁺/CD3⁺ T cell population is shown. In addition extrapolated total amounts of pMHC-I multimer⁺ cells are indicated. FIG. 11(b) Corresponding T-Plex Assay: Four different T-Plex bead pools (10,000 beads each) either loaded with cognate CMV/A2-Fc or control pMHC-I-Fc were combined with the spiked T cell sample followed by T-Plex Assay analysis. T-Plex Assay was performed in a 500 μl tube rotating at 37° C. for 4 h at 40 rpm. Shown is the IFN-γ signal of the cognate CMV/A2 T-Plex beads (blue/light grey) and one representative corresponding control bead signal (dark grey). Extrapolated total amounts of IFN-γ+T-Plex beads are shown in red numbers. Pairs of upper and lower FACS-plots represent data analysis from the same reaction/bead mix (multiplex detection). FIG. 11(c) Linear range assessment. Amounts of spiked CMV/A2 T cells derived from counting chamber-based calculations is plotted against extrapolated total amounts of pMHC-I multimer⁺ cells (left panel) or extrapolated totals amounts of IFN-γ⁺ T-Plex beads (right panel). A linear regression curve is shown (red dotted line).

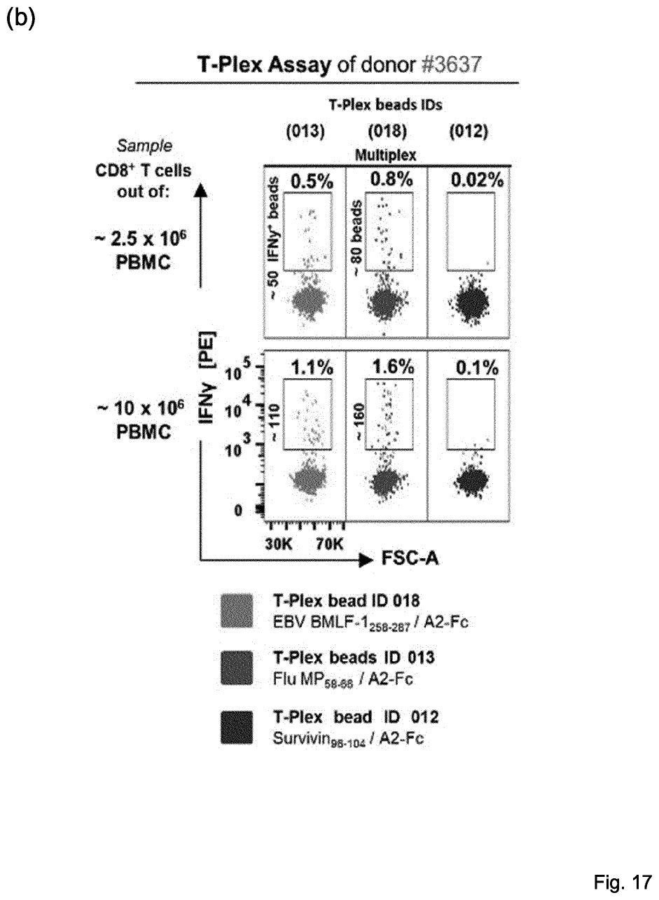

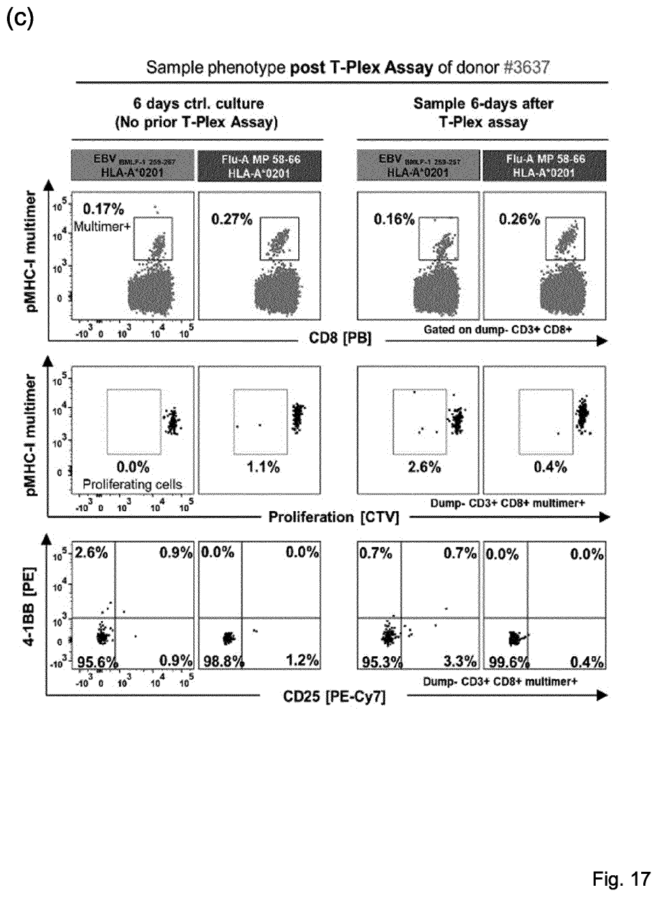

As shown in FIG. 11(a), a very low frequency of 0.005% (˜20 out of 2.5×10⁵ cells) was reliably detected by commercial HCMV pp65₄₉₅₋₅₀₃/A2-multimer (Pentamer, ProImmune) staining, which is in accordance with the published detection limit of a pMHC-I-multimer staining (Bentzen and Hadrup, Cancer Immunol Immunother. 66(5):657-666, 2017). In contrast, 100 to 200 antigen-specific T cells represent the detection limit of the T-Plex Assay, which is also shown to be largely independent of surrounding bystander T cells (see FIG. 11(b)). Thus, while the pMHC-I-multimer staining was approximately 10× more sensitive compared to the T-Plex Assay, in contrast to the T-Plex Assay, it does not allow for a functional assessment of the T cell response and/or for the recovery of the test sample. In terms of dynamic range, the presence of more than 10,000 cognate T cells led to a severe IFN-γ bystander capture onto control T-Plex beads resulting in a less distinct separation between cognate (matching APM) and control T-Plex beads (not matching APM). Indicating that, in such a setting, the T-Plex Assay's performance is somewhat similar to that of an ELISpot assay. However, for a standard 96-well plate-based ELISpot the signal saturation is already reached at 900-1000 spot-forming cells/wells resulting in a lack of distinction of single spot-forming cells (Karlsson et al., J. Immunol. Methods. 283(1-2):141-153, 2003). In contrast, the T-Plex Assay displays a linear relationship between ˜100 and 10000 cognate T cells (FIG. 11(c)). Advantageously, within this range the amount of detected IFN-γ-loaded T-Plex beads (IFN-γ⁺) reliably reflects the amount of antigen-specific T cells present in the sample. This finding not only holds true for the model T cell lines used in FIG. 11 but also for antigen-specific T cells within the entire CD8⁺ T cell pool as shown in FIG. 17/Example 8. Moreover, unlike an ELISpot assay the T-Plex Assay allows for the parallel detection of multiple antigen-specific T cell responses within a single reaction.

### Example 4: Optimization of the T-Plex Assay and Identification of Crucial Assay Parameters

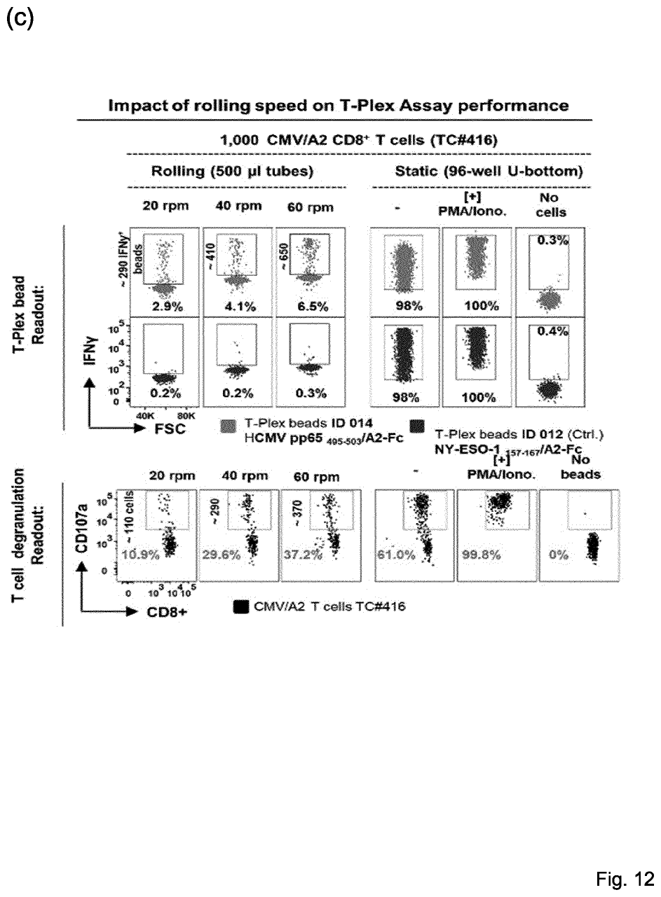

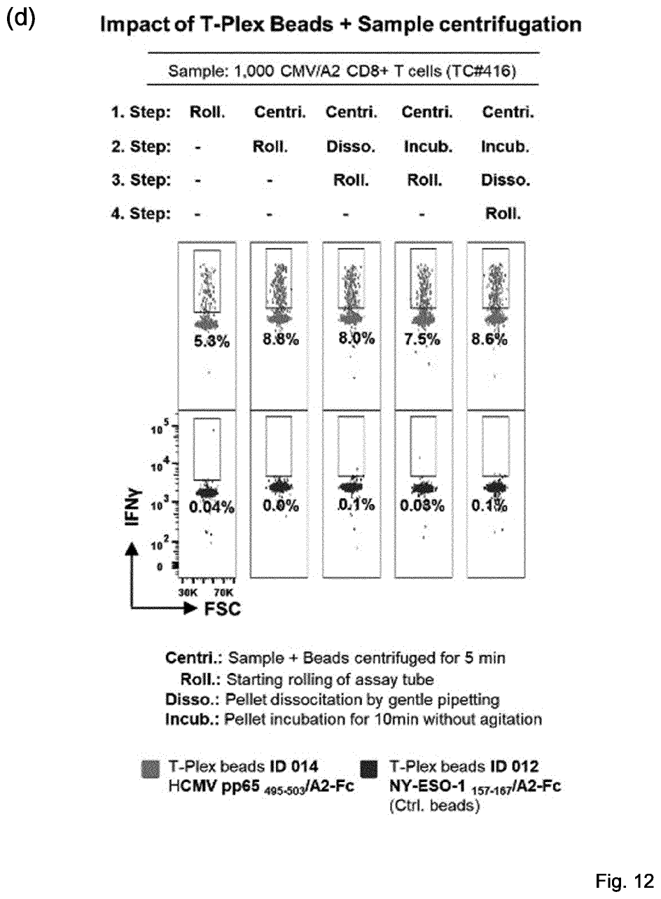

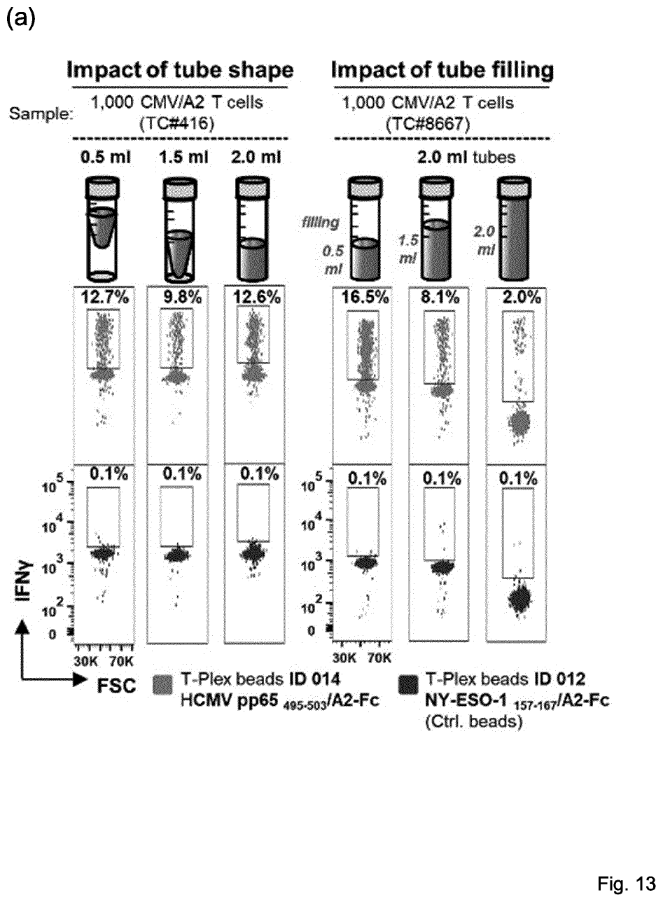

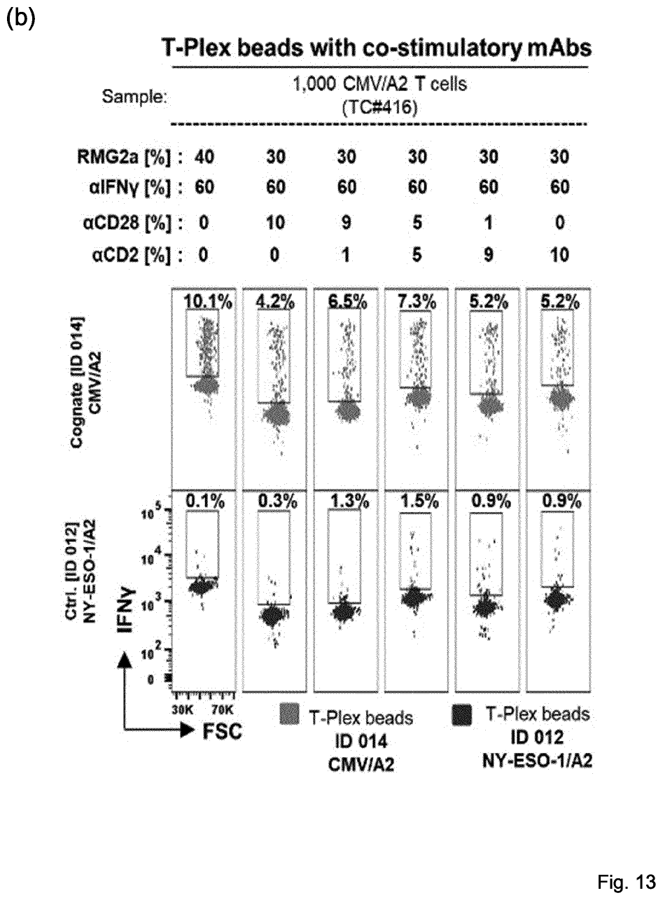

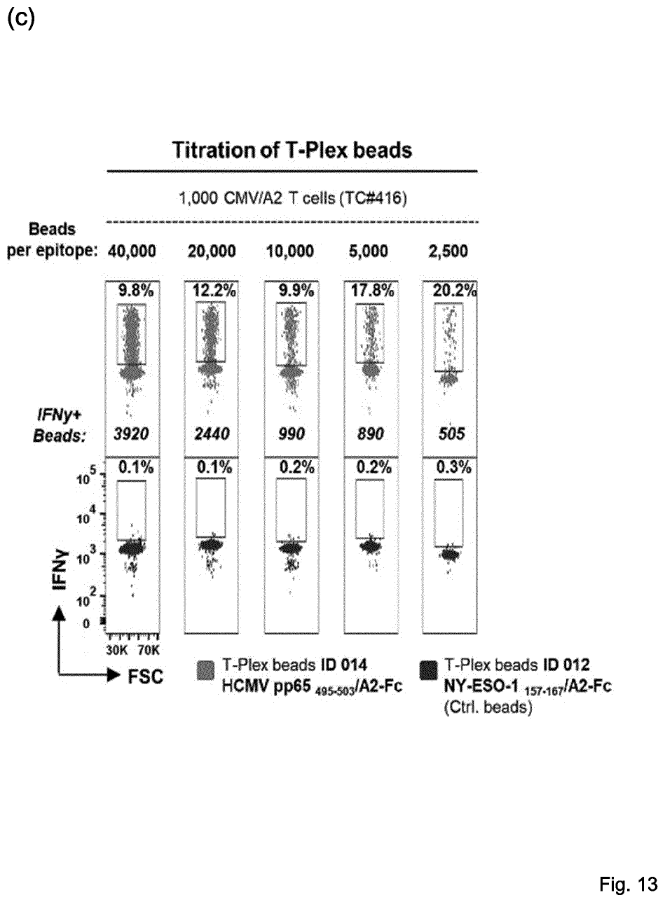

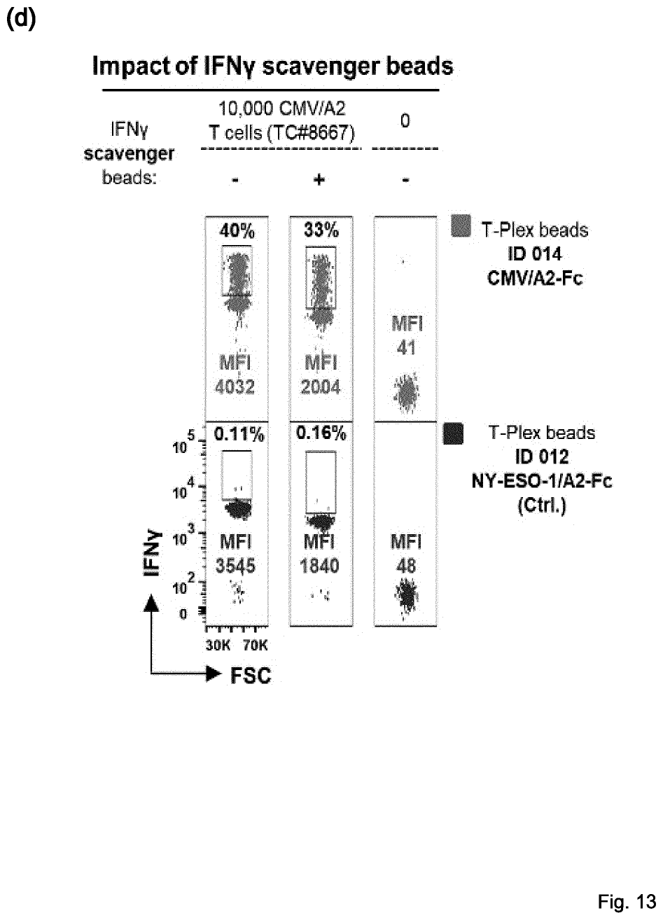

In order to improve the T-Plex Assay's sensitivity, we analysed the impact of various parameters including (1) the T-Plex bead composition itself, (2) the duration of the T-Plex reaction, (3) the tube rolling speed as well as (4) the initial proximity of test sample and T-Plex bead prior to rolling (FIG. 12). In addition, the following parameters were analysed in FIG. 13 including (5) the used assay tube with regard to shape and size, (6) presence of co-stimulatory acting antibodies on the T-Plex bead, (7) the amount of used T-Plex beads per test and (8) additional supplementation of the assay reaction with bystander beads having solely IFN-γ-capture capacity (IFN-γ scavenger beads).

In all experiments shown in FIG. 12, approximately 1,000 cells of a HCMV pp65₄₉₅₋₅₀₃/HLA-A2 (CMV/A2) specific CD8⁺ T cell line were used as test sample for the T-Plex Assay reactions. In FIGS. 12(a) and (b) the T cell line (Tc) was generated from healthy donor #8667 and in FIGS. 12(c) and (d) from #416. Unless otherwise mentioned, T-Plex beads were assembled using covalently conjugated anti-IFN-γ capture monoclonal antibody (αIFN-γ mAb) clone MD-1 (MD-1) and rat α-mouse IgG2a isotype (α-mlgG2a mAb) clone RMG2a at a 3 to 2 ratio (60% MD-1/40% Clone RMG2a). Subsequently, defined colour-coded beads (ID) were loaded with SCT-based pMHC-I-mIgG2a-Fc constructs. Assembled T-Plex beads (4× multiplex/10,000 beads each) and test sample were rotated at 40 rpm, 37° C. for 4 h. Next, T-Plex beads were stained with αIFN-γ detection mAb (clone 4S.B3) and analysed. FIG. 12(a) Titration of α-mIgG2a/pMHC-I-Fc to α-IFN-γ mAb ratio on T-Plex beads and usage of two different αIFN-γ mAbs: Shown is a representative T-Plex Assay result as FACS blot for standard 60:40 αIFN-γ/pMHC-I-Fc T-Plex beads either on the basis of the αIFN-γ clone NIB42 or clone MD-1 (BioLegend). Shown is the IFN-γ signal of the cognate CMV/A2 T-Plex beads (blue, light grey) and one-out-of-three corresponding control bead signals (dark grey). The bar diagram displays the performance of T-Plex beads depending on the ratio of anti(α)-IFN-γ to α-mIgG2a/pMHC-I-Fc on the T-Plex beads. FIG. 12(b) Kinetics of the T-Plex Assay: The T-Plex Assay was conducted as mentioned above and the reaction was stopped and analysed after the indicated incubation times. FIG. 12(c) T-Plex Assay performance based on rolling speed: T-Plex Assays were conducted at various rpm using a 500 μl tube or in static conditions using a 96-well of U-bottom plate. Degranulation of CD8⁺ T cells was analysed in parallel reactions using α-CD107a staining as described methodically by (Betts et al., J. Immunol. Methods. 281(1-2):65-78, 2003). FIG. 12(d) Impact of centrifugation of test sample and T-Plex beads prior rolling: Test sample and T-Plex beads were filled in a conical skirted 500 μl tube combined with CO₂-satured assay medium and vortexed. The next steps were performed prior to rolling as indicated.

For the experiments shown in FIGS. 13(a), (c), and (d), T-Plex beads were assembled using covalently conjugated αIFN-γ capture mAb (clone MD-1) and rat α-mlgG2a (Clone RMG2a) at a 3 to 2 ratio (60% MD-1/40% RMG2a). Subsequently, defined bead regions (ID) were loaded with pMHC-mlgG2α-Fc constructs. Unless otherwise indicated assembled T-Plex beads (4× multiplex/10,000 beads each) and test samples were rotated at 60 rpm, 37° C. for 4 h 30 min. Next, T-Plex beads were stained with αIFN-γ detection mAb and analysed. In FIG. 13 (a)-(c) ˜1,000 cells and in FIG. 13 (d) ˜10,000 of a CMV HCM pp65₄₉₅₋₅₀₃/HLA-A2 (CMV/A2) specific CD8⁺ T cell line (TC) were used as test sample for the T-Plex Assay reactions either derived from healthy donor #8667 or #416. In FIG. 13(a)-(d) the IFN-γ signal of the cognate CMV/A2 T-Plex beads is shown (blue, light grey) and one-out-of-three corresponding ctrl bead signal (dark grey).

The impact of tube shape, size and filling level on T-Plex Assay performance was assessed and the results are shown in FIG. 13(a). The T-Plex Assay was performed as described above using different kinds of tube sizes/shapes and filling levels (red) as indicated in the figure. The performance of T-Plex beads additionally supplemented with co-stimulatory mAbs was assessed and the results are shown in FIG. 13(b). Here, T-Plex beads were assembled using covalently conjugation of the indicated ratios of rat α-mlgG2a (Clone RMG2a), αIFN-γ capture mAb (Clone MD-1) and αCD28 mAb (Clone 15E8) as well as αCD2 mAb (RPA-2.10). In a second step, pMHC-I-Fc was loaded to generate fully assembled T-Plex beads. The effect of the amount of T-Plex beads was assessed by way of titration experiments and the results are shown in FIG. 13(c). The T-Plex Assay was performed using the indicated amounts of 60:40 αIFN-γ/pMHC-I-Fc T-Plex beads and 1,000 CMV/A2 T cell test sample. Extrapolated total amounts of IFN-γ⁺ T-Plex beads are shown in red. Further, the impact of IFN-γ scavenger beads on T-Plex Assay performance was assessed and the results are shown in FIG. 13(d). The T-Plex Assay was performed in the absence or presence of 2×10⁵ IFN-γ scavenger beads, which are goat-α-mlgG Dynal beads only loaded with αIFN-γ mAb (MD-1). The T-Plex Assay was performed using 10,000 CMV/A2 T cells, which represent the T-Plex Assay's outer dynamic range. Median fluorescence intensity (MFI) of the total bead population is shown.

Capture of the effector molecules secreted from an activated T cell on a proximal binding T-Plex bead is a key element for the T-Plex Assay. Here, the capture of IFN-γ or of any other suitable effector cytokine is influenced by multiple parameters. One key parameter is the selection and the intrinsic properties of the capture antibody. For IFN-γ, the clone MD-1 outperformed the NIB42 clone with regard to overall brightness and clustering of cognate IFN-γ-loaded T-Plex beads as well as the total fraction of beads that actually become IFN-γ⁺. Moreover, a 50:50 ratio of pMHC and IFN-γ capture antibody on the bead surface, an assay incubation time between 4 to 6 h and a tube filling of 500 μL medium was most favourable.

In addition, centrifugation of the test sample and T-Plex beads prior to rolling at 60-80 rpm improves assay sensitivity. In sharp contrast, static conditions meaning incubation of a test sample with T-Plex beads in a 96-well plate without movement, leads to an indistinguishable bystander IFN-γ-loading on all T-Plex bead pools independent of their linked APM. Coupling of co-stimulatory antibodies (i.e. anti-CD28 antibodies) and reducing the amount of pMHC coupled to the T-Plex bead decreased the assay performance. The usage of more than 10,000 T-Plex beads per T cell epitope might slightly increase the assay sensitivity. However, 10,000 T-Plex beads/T cell epitope have proven as robust allowing for the enumeration of the antigen-specific T cell population, since substantive linearity between 200 and 10,000 antigen-specific T cells can be observed. This dynamic range may be further improved by the additional usage of beads, which display a high IFN-γ-capture capacity but lack a T cell stimulation capacity (i.e. lack of an APM referred to as IFN-γ scavenger beads) as they can reduce the overall bystander (antigen-independent) IFN-γ loading onto all T-Plex beads present during the co-culture.

### Example 5: aAPC Assembly Variations and Impact on T-Plex Assay Performance

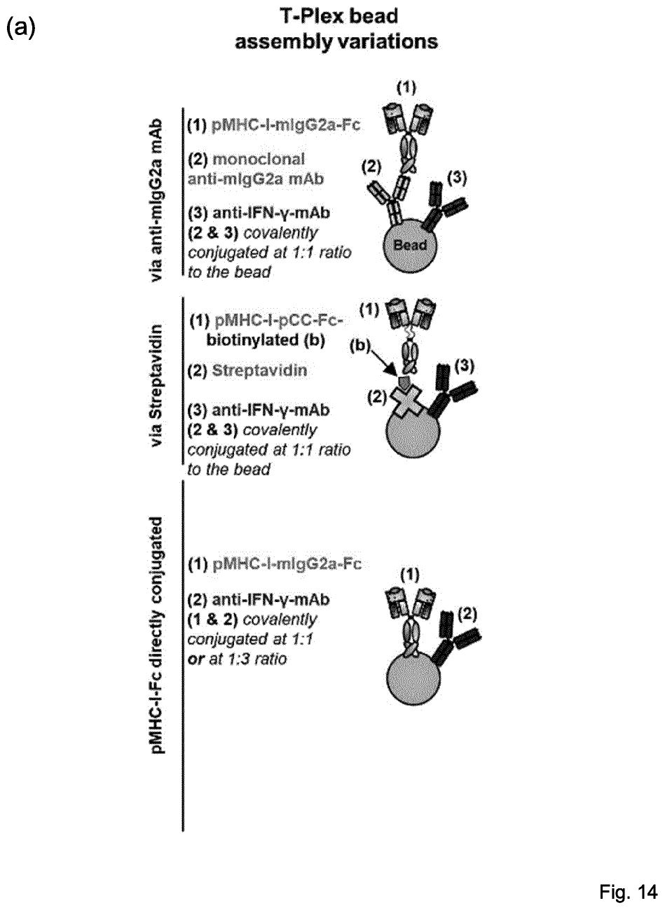

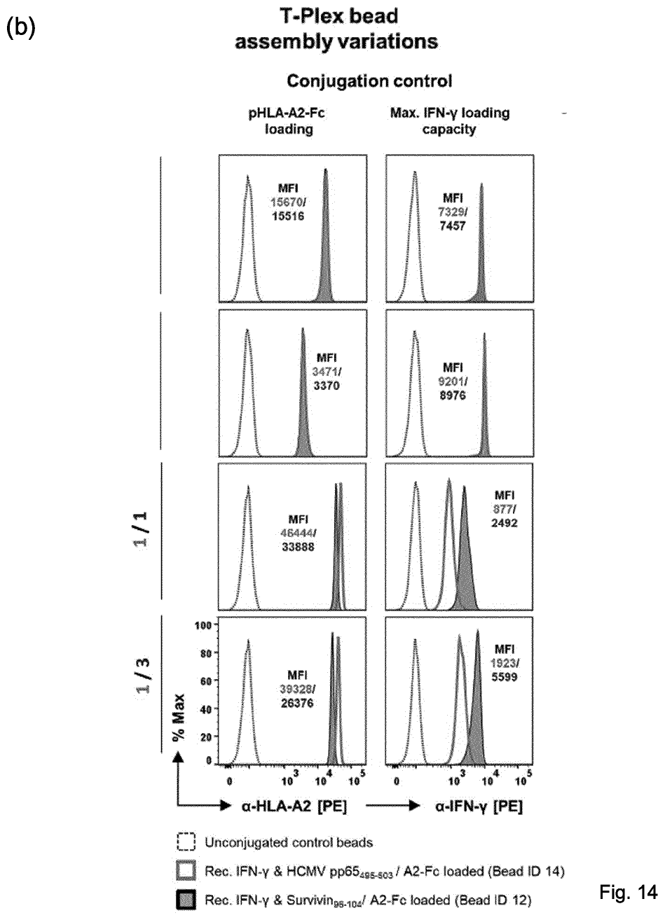

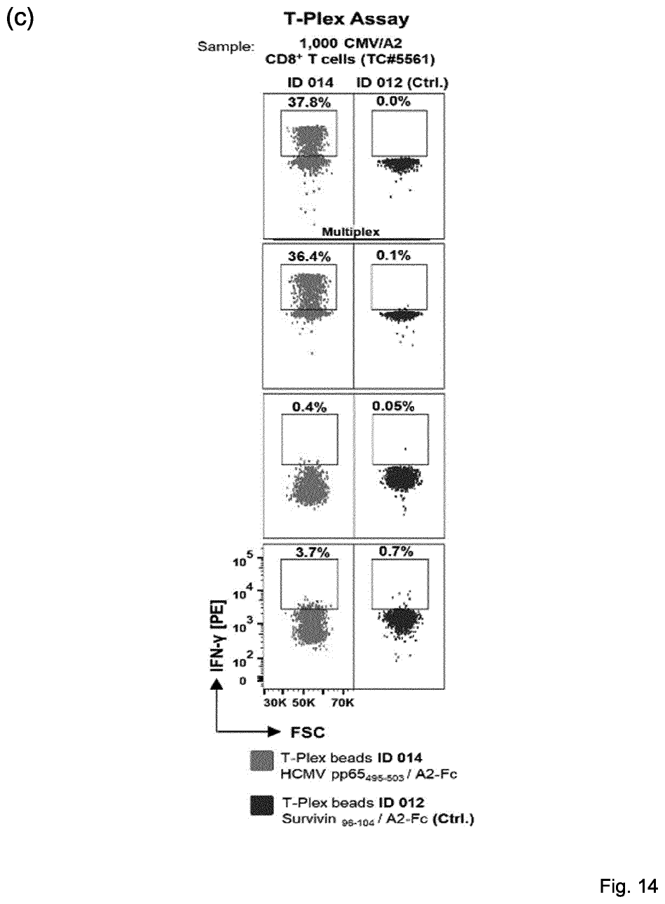

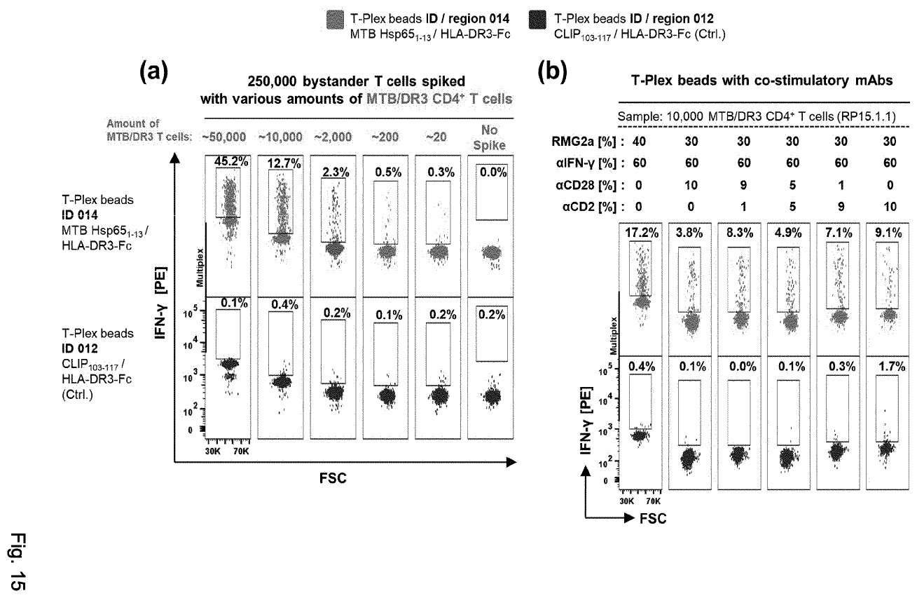

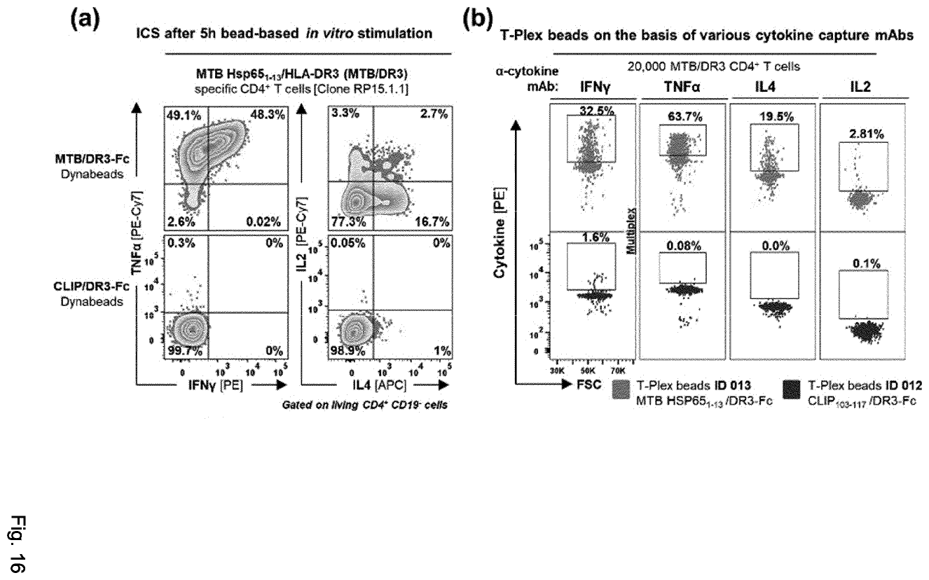

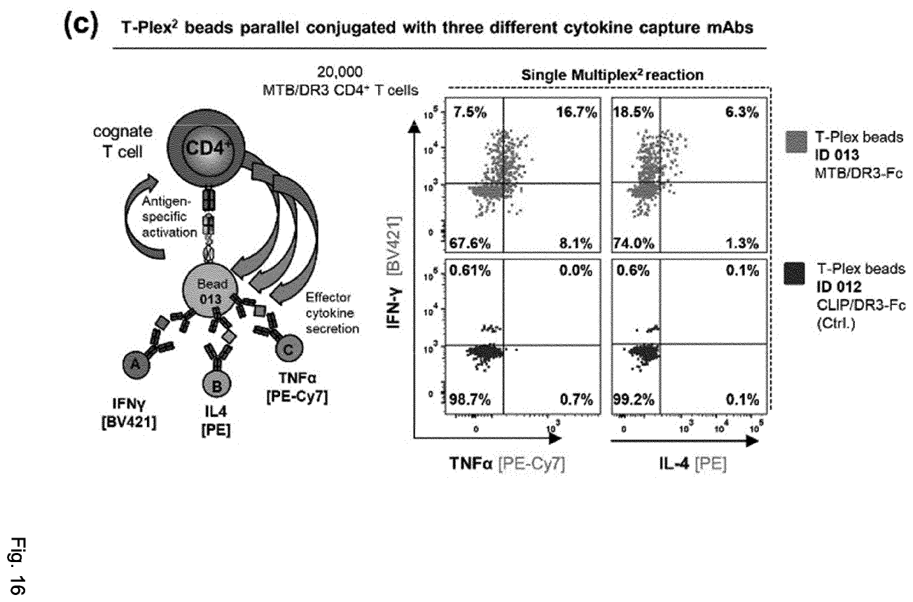

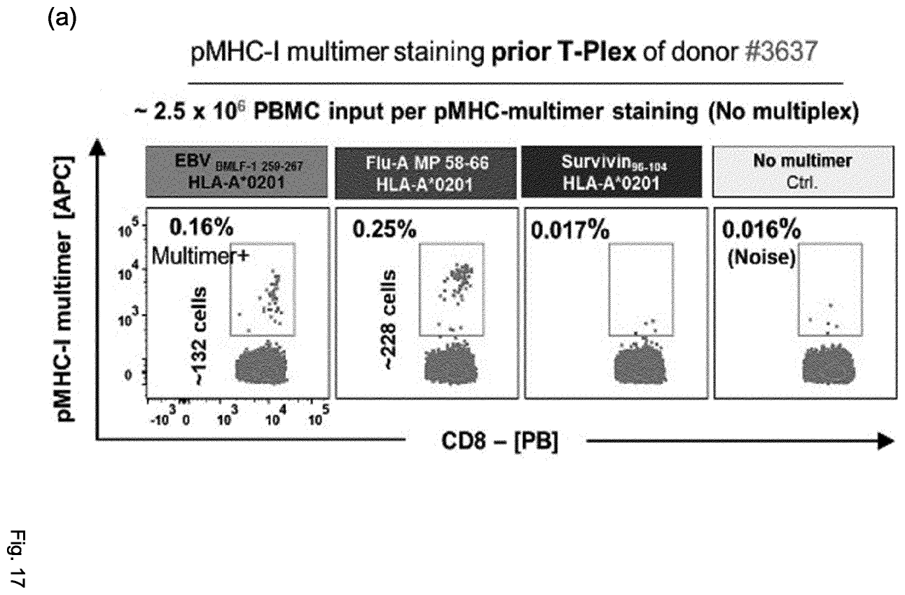

FIG. 14(a) provides a scheme of aAPC (T-Plex bead) assembly variations. To obtain the data shown in the upper panel of the Figure, T-Plex beads as previously described were used, with the modification that colour-coded Luminex beads (Bead) were covalently conjugated with αIFN-γ capture mAb (Clone MD-1) and rat α-mlgG2a (Clone RMG2a) in a 1 to 1 (50% to 50%) ratio. Subsequently defined bead regions (ID) were loaded with soluble pMHC-I-mIgG2a-Fc aAPMs [CMV/A2-Fc and Survivin/A2-Fc] (as also illustrated in FIG. 1) constructs derived from supernatants of construct expressing CHO-S cells. To obtain the data shown in the middle panel of the Figure, Luminex beads were covalently conjugated with streptavidin and αIFN-γ capture mAb (Clone MD1) in a 1:1 ratio and subsequently loaded with purified biotinylated pMHC-I-pCC-mIgG2a-Fc-Bio (also shown in FIG. 3). To obtain the data shown in the bottom panel, Luminex beads were directly covalently conjugated with purified pMHC-I-Fc-STag constructs and αIFN-γ capture mAb either in a 50% to 50% (1:1) or 25% to 75% (1:3) ratio. FIG. 14(b) provides an indication of corresponding conjugation quality control. pHLA-A2 conjugation was analysed by staining of final pHLA-A2 loaded/conjugated T-Plex beads with αHLA-A2 mAb (Clone BB7.2/BioLegend) of IgG2b isotype. Maximal αIFN-γ-capture capacity was analysed by incubating fully-assembled T-Plex beads with 4 ng/ml recombinant IFN-γ (BioLegend) for 2 h at 37° C. followed by αIFN-γ-PE detection mAb (Clone 4S.B3/BioLegend). Shown is the fluorescence signal of the HCMV pp65₄₉₅₋₅₀₃/HLA-A2-Fc (CMV/A2-Fc) (blue, light grey) and Survivin₉₆₋₁₀₄/HLA-A2-Fc (Survivin/A2-Fc) conjugated T-Plex beads as well as unloaded Luminex beads (dotted line). FIG. 14(c) illustrates corresponding T-Plex Assay performance. T-Plex Assay was performed using the in (a) indicated T-Plex bead assembly variations. 1000 CMV/A2 specific T cells (TC #5561) were combined T-Plex beads (4× multiplex/10,000 beads per T cell epitope) in a 500 μl tube and centrifuged prior rolling at 60 rpm for 4 h at 37° C. Shown is the IFN-γ signal of the cognate CMV/A2 T-Plex beads (blue, light grey) and one-out-of-three corresponding control bead signals (dark grey). Rows of T-Plex Assay FACS plots represent data analysis from the same reaction/bead mix (multiplex detection).

Luminex beads covalently conjugated either with a 50:50 ratio of α-mlgG2a-Fc (α-Fc) mAb [RMG2a]/IFN-γ-capture mAb [MD-1] or streptavidin [SAv]/IFN-γ-capture mAb [MD-1] and subsequently loaded with pHLA-A2-Fc or biotinylated pHLA-A2-pCC-Fc displayed very similar pMHC-Fc binding and maximal IFN-γ-capture capacities. However, staining for HLA-A2 (clone BB7.2, IgG2b isotype) of pMHC-Fc-loaded α-Fc-based T-Plex beads revealed roughly 4-fold higher median fluorescent intensity (MFI) values compared to pMHC-pCC-Fc-loaded SAv-based T-Plex beads indicating a slightly higher pMHC-binding capacity of the α-Fc-based T-Plex beads. Nevertheless, a T-Plex Assay for the detection of 1,000 CMV/A2 specific CD8⁺ T cells (TC #5561) using either α-Fc-based or SAv-based T-Plex beads performed almost equally. Thus, the differing T-Plex bead architectures (i.e. on the basis of either IFN-γ-capture mAb/α-mlgG2a-Fc mAb beads or IFN-γ-capture mAb/streptavidin beads subsequently loaded with pMHC-Fc or biotinylated pMHC, respectively, were shown to provide advantageous and overall very similar T-Plex Assay performances.

In order to distinguish antigen-dependent IFN-γ-loaded T-Plex beads (i.e. an aAPC that induced a cognate T cell response) from bystander IFN-γ-loaded T-Plex beads (i.e. aAPCs with a non-matching APM present during the co-culture of the entire group of aAPCs and T cells) it is key, that cognate and control T-Plex beads have the same IFN-γ-binding capacity. This is easily achieved, if multiple Luminex beads of different colour but with homogenous protein-binding capacity are covalently conjugated and used as a “production batch” with a shared master mix comprising the IFN-γ-capture mAb and streptavidin or, alternatively, an α-mlgG2a-Fc mAb.

### Example 6: Antigen-Specific Detection of MTB/DR3 CD4+ T Cell Clone RP15.1.1 by pMHC-II-Fc Loaded T-Plex Beads

FIG. 15 shows an T-Plex Assay based on pMHC-II for the detection of a single CD4+ T cell line (model system). In particular, the detection of Mycobacterium tuberculosis (MTB) heat-shock protein 65 (Hsp65)₁₋₁₃/HLA-DRB1*03:01/DRA*01 (MTB/DR3)-specific CD4⁺ T cell line clone RP15.1.1 by the T-Plex Assay is shown in FIG. 15(a). T-Plex beads (60% αIFN-γ mAb [Clone MD-1]/rat α-mlgG2a 40% [Clone RMG2a]) were loaded with MTB Hsp65₁₋₁₃/HLA-DR3-pCC-Fc (purple (light grey)/cognate) or CLIP₁₀₃₋₁₁₇/DR3-pCC-Fc (dark grey/control) also described in FIG. 2. Subsequently, pMHC-II-Fc-loaded T-Plex beads (2× multiplex/10,000 beads each) were combined with indicated amounts (red numbers) of the MTB Hsp65₁₋₁₃/HLA-DR3 specific CD4⁺ T cell clone RP15.1.1 (MTB/DR3 T cells) in the presence of 250,000 Survivin₉₆₋₁₀₄/HLA-A2 specific CD8⁺ T cells (bystander T cells). The T-Plex Assay was performed in 500 μl tubes, rolling at 60 rpm, 37° C. for 5 h. Next, T-Plex beads were stained with αIFN-γ detection mAb and analysed. The presence of antigen-specific T cells was indicated by appearance of an IFN-γ⁺ subpopulation of cognate T-Plex beads that is above T-Plex control beads (dark grey). Pairs of upper and lower T-Plex Assay FACS plots represent data analysis from the same reaction/bead mix (multiplex detection). FIG. 15(b) illustrates the performance of T-Plex beads supplemented with co-stimulatory mAbs. Here, T-Plex beads were assembled using covalently conjugation of the indicated ratios of rat α-mlgG2a (RMG2a), αIFN-γ capture mAb (Clone MD-1) and αCD28 mAb (Clone 15E8) as well as αCD2 mAb (RPA-2.10). In a second step, pMHC-II-Fc were loaded to generate fully assembled T-Plex beads and which were finally combined with 10,000 MTB/DR3 T cells and the T-Plex Assay was performed as described above.

Cognate MTB/DR3-associated T-Plex beads were used to reliably detect the presence of 50,000 to 200 MTB/DR3-specific CD4⁺ T cells (FIG. 15(a)). However, the resulting fractions of IFN-γ⁺ T-Plex bead populations were somewhat lower as seen in similar experiments using the CMV/A2 specific CD8⁺ T cell line #416 or #5561. This can be explained by the fact that only 40-60% of the MTB/DR3-specific CD4⁺ T cell actually express IFN-γ upon stimulation with bead-based aAPCs. Moreover, T-Plex beads additionally supplemented with co-stimulatory antibodies decreased the detection performance of the MTB/DR3-specific CD4⁺ T cells line (FIG. 15(b)), which was in accordance with previous T-Plex Assay optimization experiments presented in FIG. 13(b). In conclusion, the inventors show that the T-Plex Assay concept-based on T-Plex beads can also be applied for the antigen-specific detection of IFN-γ-secreting Tₕ1-differentiated CD4⁺ T cells.

### Example 7: Proof-of-Principle of a T-Plex² Assay for the Antigen-Specific Detection of CD4+ T Cells

The T-Plex² (Detecting multiple cytokines on the same bead) proof-of-concept data of this example illustrates that: a) using an independent assay, the CD4+ T cell line produces multiple cytokines upon stimulation with aAPCs of the present invention; b) the assay of the third aspect of the present invention (T-Plex Assay) also works on the basis of cytokines TNFa, IL-4 and IL-2; and c) the assay of the fourth aspect of the invention (T-Plex² Assay) is suitable for the detection of several levels of multiplexation, namely the detection of multiple T cell specificities (2 in this example) and multiple cytokines (3 in this example).

FIG. 16(a) shows cytokine intracellular staining (ICS) after 5 h aAPC-based re-stimulation of MTB/DR3 CD4⁺ T cell line: MTB Hsp65/HLA-DR3-pCC-Fc (MTB/DR3-Fc) (cognate) and CLIP₁₀₃₋₁₁₇/DR3-pCC-Fc (CLI P/DR3-Fc) coated goat-α-mouse-IgG-Fc Dynabeads (Invitrogen) were co-cultured with equal amounts of MTB Hsp65₁₋₁₃/HLA-DR3-specific CD4⁺ T cell clone RP15.1.1 (MTB/DR3 T cells) for 5 h at 37° C. in a 96-well U-bottom in the presence of brefeldin A and monensin to block cytokine secretion. The frequency of cytokine expressing cells within the CD4⁺ T cell population is shown. FIG. 16(b) shows the detection of different cytokines by the T-Plex beads platform: T-Plex beads were assembled using covalently conjugated αIFN-γ capture mAb (Clone MD-1) and monoclonal α-mlgG2a at a 60% to 40% ratio. Alternatively, the αIFN-γ was replaced by cytokine capture mAbs binding to IL-2 (clone MQ1-17H12), IL-4 (clone 8D4-8) or TNF-α (clone Mab 1). Subsequently, pMHC-II-loaded T-Plex bead pools on the basis of different cytokine mAb were loaded with MTB/DR3-Fc (purple (light grey)/cognate) or CLIP/DR3-Fc (dark grey/control) and finally combined with MTB/DR3-specific CD4⁺ T cells. The assay was performed in 500 μl tubes, rolling at 60 rpm, 37° C. for 5 h using 10,000 beads each (2× multiplex). After the T-Plex reaction, the T-Plex beads were stained with the corresponding cytokine detection mAbs (all conjugated to the fluorochrome phycoerythrin (PE)) and finally analysed by FACS. Pairs of upper and lower T-Plex Assay FACS-plots represent data analysis from the same reaction/bead mix. FIG. 16(c) illustrates the T-Plex² Assay proof-of-principle data: T-Plex² beads were assembled using covalently conjugated αIFN-γ, αTNF-α and IL-4 capture mAbs (each 1/5 (20% of the total Luminex bead protein binding capacity) and monoclonal α-mlgG2a (2/5 [40%]). pMHC-II-loaded T-Plex² beads were incubated with the MTB/DR3-specific T cells and T-Plex Assay was performed as described above. Next, T-Plex² beads were stained with the corresponding cytokine detection mAbs conjugated to different fluorochromes (Brilliant violet 421 nm (BV421), PE and PE/Cy7) as indicated in the figure and analysed by FACS. All four T-Plex Assay FACS plots shown are from the same T-Plex² reaction/bead mix.

Upon co-culture with the MTB/DR3-specific CD4⁺ T cell clone, cognate T-Plex² beads were partially loaded with a combination of effector cytokines (IFN-γ, TNF-α and IL-4) which could be simultaneously detected in a single multi-dimensional multiplex reaction. Thus, T-Plex² beads are suitable to provide a functional profile of the MTB/DR3-specifc CD4⁺ T cell clone similar to the ICS data, which represents a proof-of-principle for the T-Plex² concept.

### Example 8: Antigen-Specific T Cell Detection Using the T-Plex Assay does not Change the Phenotype of the Original Sample

The data of this example, as also illustrated in FIG. 17, illustrates what effects performing an assay of the present invention on a sample of T cells has. As is further described directly below, a sample, which was also in parallel characterized by pMHC-multimer analysis, was analysed by the T-Plex Assay (beads were analysed by FACS) and the T cells were brought back into culture. After 6 days, the phenotype of the sample, which were previously subjected to analysis by the T-Plex Assay, was compared to that of an “untouched” sample of corresponding T cells, which had been cultured also in parallel for 6 days. No obvious changes/differences between the phenotypes of those samples were observed. Thus, subjecting a T cell population to analysis by the T-Plex Assay induces hardly any phenotypic changes to the cells of the sample.

In FIG. 17(a) pMHC-I multimer staining of healthy donor (HD) sample #3637 is shown: A T cell sample from HLA-A2⁺ healthy donor (HD) #3637 was initially analysed by pMHC-I multimer staining (no multiplex). The frequencies of pMHC-I multimer+ cells within the CD8⁺ T cell population are shown in black/horizontal numbers. Extrapolated total amounts of respective antigen-specific CD8⁺ T cells within 2.5×10⁶ PBMC are shown in red/vertical numbers. In FIG. 17(b) data on corresponding T-Plex Assay-based analysis of a T cell sample from the same donor is shown: To analyse if a T-Plex run induces antigen-specific proliferation, PBMC of HD #3637 were labelled with CellTrace violet (CTV/Invitrogen) prior isolation of untouched CD8⁺ T cells. pMHC-I-Fc loaded T-Plex bead-pools (4× multiplex (CMV/A2; Flu/A2; EBV/A2; Survivin/A2)/10,000 beads each) were incubated with isolated CD8⁺ T cells derived from either 2.5×10⁶, 10×10⁶ total PBMC in 500 μl tubes. Prior to rolling at 60 rpm for 5 h, 37° C., the test sample and T-Plex beads were centrifuged for 5 min at 1500 rpm. After 5 h T-Plex beads were magnetically separated from the T cells and analysed. T cell sample was brought back into culture for 6 days together. In FIG. 17(c) illustrates sample phenotype after a T-Plex Assay run in comparison to an untouched control T cell culture. For 6 days cultured OW-labelled HD #3637 CD8⁺ T cells either previously subjected to a T-Plex Assay or left untouched (no T-Plex Assay) were additionally stained with pMHC-I multimers prior lineage and activation marker staining. FIG. 17(c), upper panel, illustrates the frequencies of pMHC-I multimer+ cells within the CD8⁺ T cell population are shown. FIG. 17(c), middle and bottom panels indicate the percent of proliferation (CTV^{dim}) and activation marker (CD25⁺/4-1BB⁺) expression of pMHC-I multimer⁺ CD8⁺ T cells.

### Example 9:Successful Eukaryotic Cell-Based Production and Antigen-Specific Binding of Soluble pMHC-I-Fc Molecules

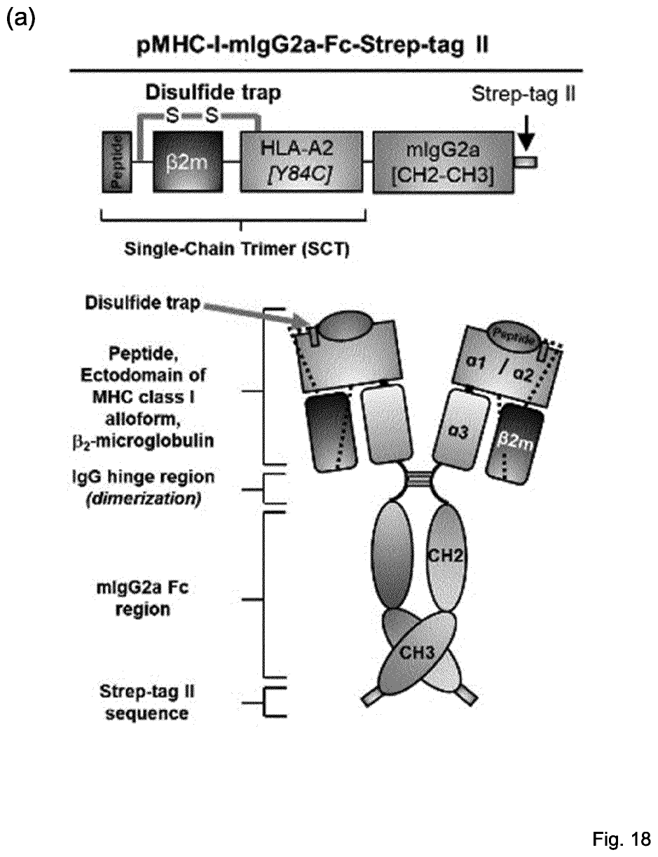

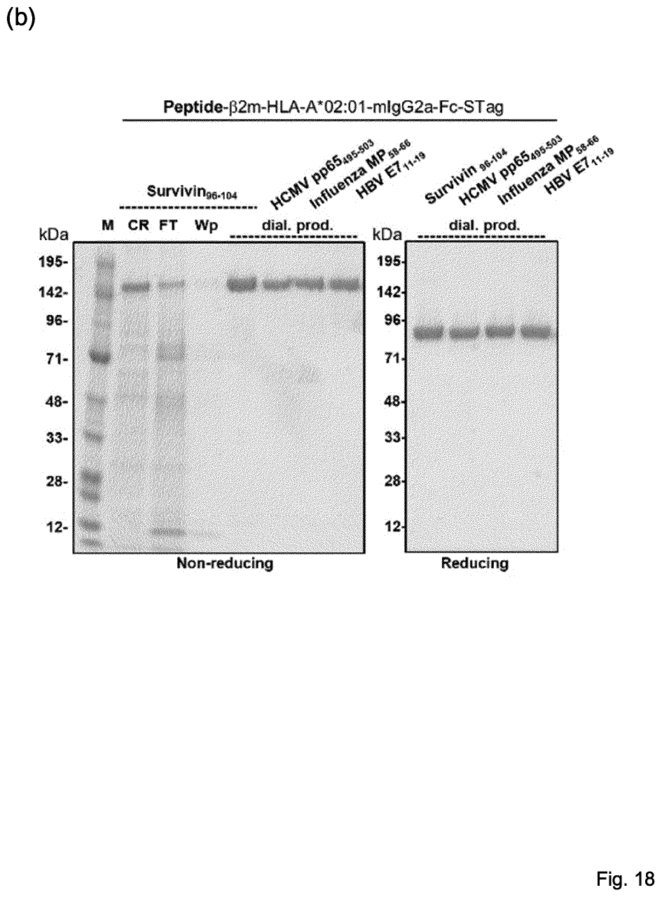

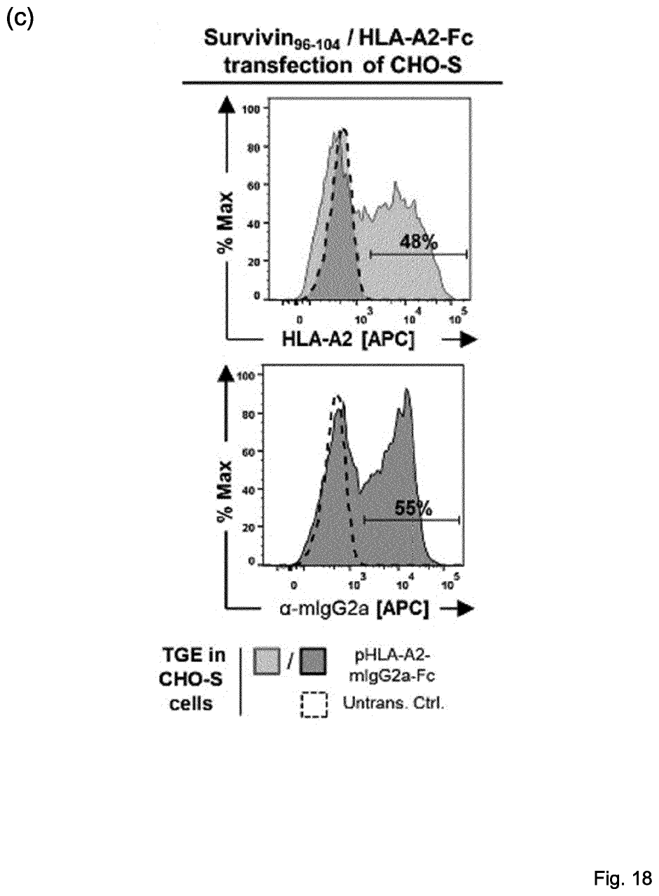

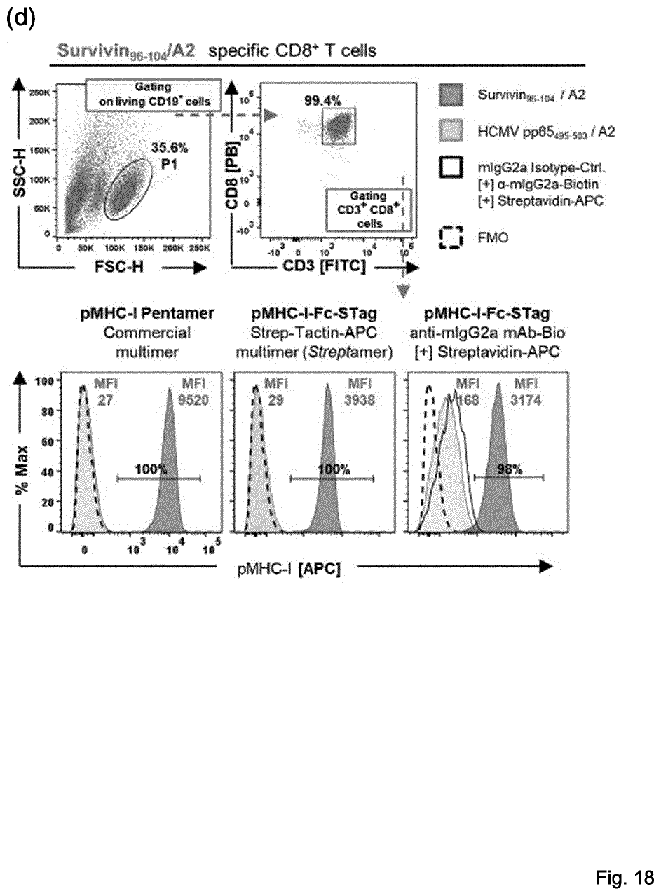

As shown in FIG. 18(a) the dimeric disulfide-trapped (dt) peptide-MHC-Class I immunoglobulin Fc fusion molecule (pMHC-I aAPM) consists of two single polypeptide chains each comprising a covalently linked T cell epitope peptide ligand (9-10 amino acids), human β₂-microglobulin (β2m), HLA-class I allele ectodomain and constant heavy chain (CH) domains 2 and 3 of murine immunoglobulin isotype IgG2a. Dotted lines indicate flexible glycine-serine linkers. The intramolecular disulfide trap between the C-terminal peptide extension and a cysteine (C) residue replacing MHC-I tyrosine (Y) 84 residue provides further stabilization of the pMHC complex. The C-terminal Strep-Tag II (STag) sequence allows affinity-purification under neutral conditions using Strep-Tactin (from IBA Lifesciences). In FIG. 18(b) results of the validation of dt-pHLA-A2-Fc expression and structural conformation are provided. Survivin₉₆₋₁₀₄/HLA-A2-Fc-STag expressing CHO-S cells were intracellularly stained either with α-HLA-A2 mAb (clone BB7.2) (blue, light grey) or with α-mlgG2a mAb (Clone RMG2a) [dark grey] 3 days after transfection. FIG. 18(c) shows the results of dt-pHLA-A2-Fc-STag affinity chromatography. CHO-S supernatant of 6 days transient pMHC-Fc-STag expression was purified using Strep-Tactin high-capacity resin filled columns at pH 7.4. 10% SDS-PAGE under non-reducing and reducing conditions after Coomassie staining. M: marker; CR: crude/CHO-S supernatant; FT: Flow-through; Wp: pooled buffers used for washing; dial. prod; dialysed product 2.5 μg/lane. FIG. 18(d) shows the results of the validation of antigen-specific binding of dt-pHLA-A2-Fc-STag proteins. Survivin₉₆₋₁₀₄/HLA-A2 specific CD8⁺ T cell line was stained in the presence of dasatinib [50 nM] with cognate [red/dark grey] and ctrl. (blue, light grey) pMHC-I multimers at 25 μg/ml followed by lineage marker staining. (Left panel) Commercial pHLA-A2 pentamer (from ProImmune); (Middle panel) dt-pHLA-A2-Fc-STag in complex with allophycocyanin-conjugated (APC) Strep-Tactin (IBA Lifesciences); (Right panel) dt-pHLA-A2-Fc-STag and sequential staining with biotinylated α-mlgG2a mAb (α-mlgG2a-Biotin) and streptavidin-APC. To exclude dead cells, T cells were labelled with ZombieAqua (BioLegend) prior to multimer staining. MFI: Median fluorescent intensity; FMO: fluorescence-minus-one background ctrl.

### Example 10: Successful Production and Antigen-Specific Binding Validation of Soluble pMHC-II-Fc Molecules

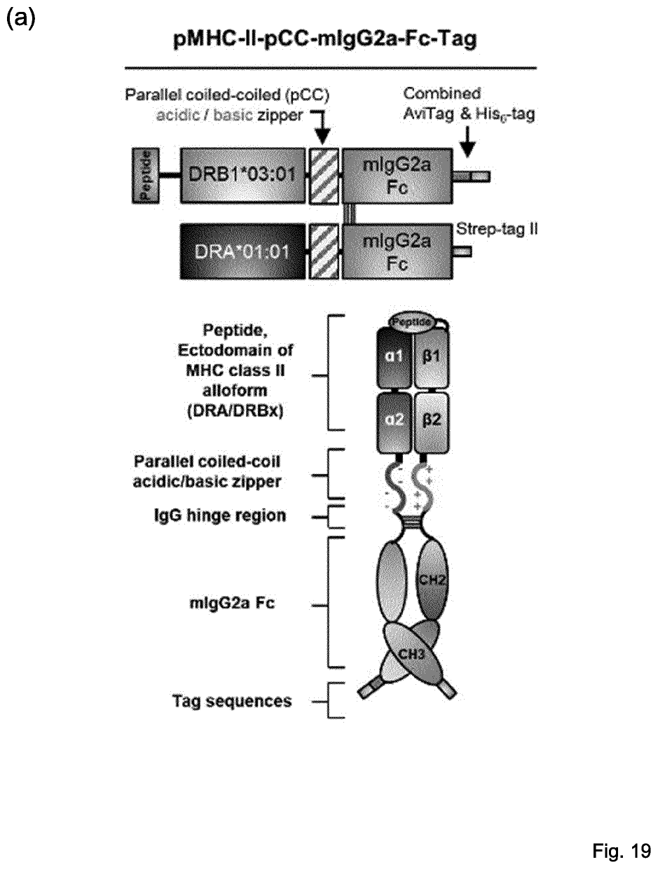

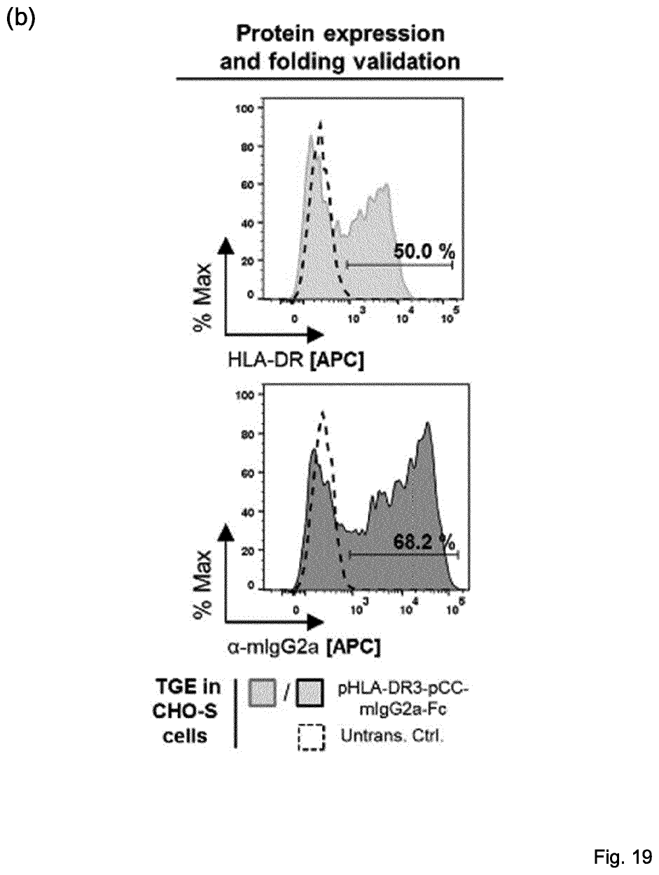

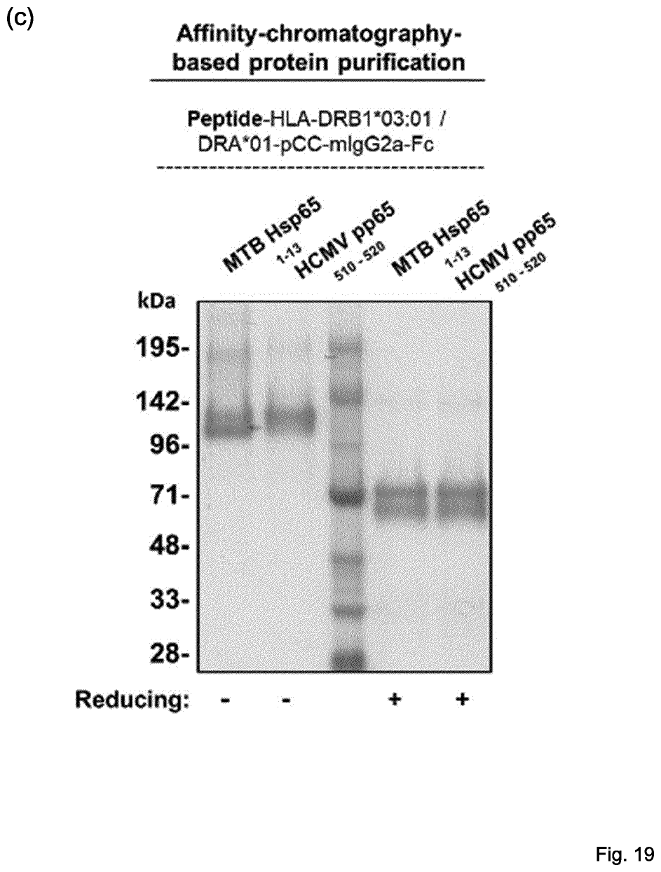

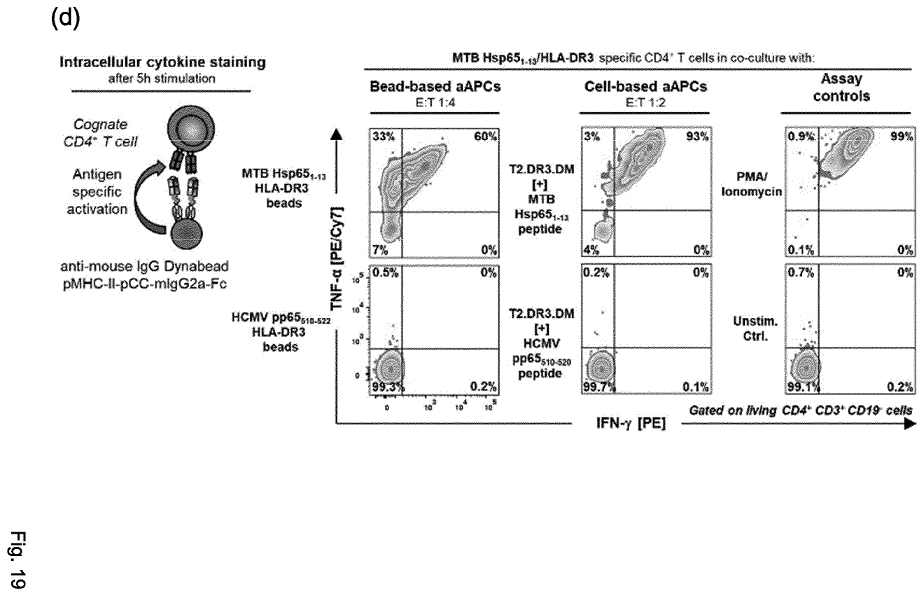

As shown in FIG. 19(a) peptide-MHC-Class II monomer immunoglobulin Fc fusion constructs (pMHC-II aAPMs) consist of two separate polypeptide chains. The MHC-II β-chain is N-terminally fused with an antigenic peptide via a flexible glycine-serine linker. The C-terminus of β-chain ectodomain 031-β₂) is fused to a parallel coiled-coil (pCC) basic zipper followed by the hinge domain and CH2 and CH3 of mlgG2a (Fc) and a C-terminal His₆_ag and AviTag for site-specific biotinylation. The ectodomain of the monomorphic α-chain (a1-α2) is C-terminally fused to a complementary acidic pCC-Fc and a C-terminal StrepTag-11. In FIG. 19(b) results of the validation of pHLA-DR3-Fc expression and structural conformation are provided. MTB Hsp65₁₋₁₃/HLA-DRB1*03:01/DRA*01 (MTB/DR3)-Fc-His/Avi/STag expressing CHO-S cells were intracellularly stained either with α-HLA-DR mAb (Clone L243) [Orange, light grey] or with α-mlgG2a mAb (Clone RMG2a) [dark grey] 3 days after transfection. FIG. 19(c) shows the results of pHLA-DR3-Fc affinity chromatography. CHO-S supernatant of 6 days transient pDR3-Fc-STag expression was purified using Strep-Tactin high-capacity resin filled columns at pH 7.4. 10% SDS-PAGE under non-reducing and reducing conditions after Coomassie staining. FIG. 19(d) shows the results of the validation of MTB/DR3-Fc specificity and bead-bound stimulatory capacity using a cognate CD4+ T cell clone. Result of a 5 h co-culture of MTB/DR3 specific CD4+ T cell clone RP15.1.1 with bead- or cell-based aAPCs are shown. Goat-α-mouse IgG-Fc-(GαM) Dynabeads previously loaded with MTB/DR3-Fc or CMV pp65610-622/DR3 (CMV/DR3-Fc, control) were used as bead-based aAPCs. HLA-DR3 and HLA-DM expressing T2 cells (T2.DR3.DM) were pulsed overnight with 10 μM MTB Hsp65₁₋₁₃ peptide or HCMV pp65₅₁₀₋₅₂₂ as control and used as cellular aAPCs. Stimulation of the MTB/DR3 CD4+ T cell clone RP15.1.1 is shown by induction of cytokine expression analysed by intracellular staining for TNF-α and IFN-γ after lineage marker staining.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An artificial antigen-presenting cell (aAPC) for the detection of effector molecules of a T cell in response to presentation of an antigen peptide sequence, the aAPC comprising:
(a) a surface, wherein the surface is the surface of a particle, optionally the surface is the surface of a bead;
(b) one or more artificial Antigen Presenting Molecules (aAPMs), wherein the aAPMs are attached to the surface via attachment sequences or via direct chemical conjugation; and
(c) one or more capture molecules attached to the surface,
wherein each of the one or more aAPMs comprises an identical antigen peptide sequence.

2. The aAPC according to claim 1, wherein the one or more capture molecules are one or more capture antibodies specific for one or more effector molecules released from a T cell in response to presentation of the antigen peptide sequence.

3. The aAPC according to claim 1, wherein the particle is coded to be identifiable and separable from other particles and, wherein, optionally,
the particle is colour-coded and the colour code is indicative of the antigen peptide sequence of the aAPM attached to the aAPC, or
the particle is magnetic.

4. The aAPC according to claim 1, wherein the aAPM is:
a monomeric, dimeric or multimeric molecule comprising soluble antigenic peptide-loadable or peptide loaded MEW class I or MEW class II portions, or soluble MEW class I or class II portions, such as a tagged recombinant soluble MHC-I molecule assembled with β₂-microglobulin and a synthetic peptide; or
a tagged recombinant soluble MHC-I molecule covalently linked with β₂-microglobulin and assembled with a synthetic peptide; or
an aAPM being a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigen peptide sequence.

5. The aAPC according to claim 4, wherein
(a) the dimerization domain comprises an IgG hinge region, optionally the IgG hinge region comprises of SEQ ID NO: 1 or SEQ ID NO: 2, and/or
(b) the Ig Fc domain is a mouse IgG2a Fc region containing an N297Q mutation or is a human IgG1 Fc region containing an N297Q mutation, optionally the IgG Fc region comprises SEQ ID NO: 3 or SEQ ID NO: 4, and/or
(c) the attachment sequence is a peptide tag for attaching the aAPM to a surface, optionally the peptide tag allows for attachment of the aAPM to the surface via affinity-based binding and/or conjugation to the surface.

6. The aAPC according to claim 4, wherein the antigen peptide sequence is a peptide sequence of an antigen selected from the group of consisting of: viral antigens; bacterial antigens; fungal antigens; parasite antigens; autoimmune; allergy-related and tumour antigens, wherein, optionally,
the antigen peptide sequence is selected from the group consisting of: HCMV pp65 495-503 (SEQ ID NO: 10); EBV BMLF-1 259-267 (SEQ ID NO: 13); Influenza virus 1VIP 58-66 (SEQ ID NO: 14), NY-ESO-1 157-165/165V (SEQ ID NO: 11); and Survivin 96-104/97M (SEQ ID NO: 12).

7. The aAPC according to claim 4, wherein the antigen-presenting domain of the aAPM comprising a single polypeptide sequence comprises in amino-to-carboxy terminal order: the antigen peptide sequence and an MHC class II portion.

8. The aAPC according to claim 7, wherein the dimerization domain of the aAPM comprising a single polypeptide sequence further comprises a parallel coiled-coiled acidic or basic zipper motif, optionally comprising a basic zipper motif of SEQ ID NO: 53 or an acidic zipper motif of SEQ ID NO: 54.

9. The aAPC according to claim 4, comprising a dimer comprising an aAPM, wherein
(a) the dimer is a homodimer or a heterodimer of two aAPMs, wherein
the aAPM comprises soluble antigenic peptide-loadable or peptide loaded MHC class I or MHC class II portions, or soluble MHC class I or class II portions, such as a tagged recombinant soluble MHC-I molecule assembled with β₂-microglobulin and a synthetic peptide, or
the aAPM comprises a tagged recombinant soluble MHC-I molecule covalently linked with β₂-microglobulin and assembled with a synthetic peptide, or
the aAPM is a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain comprises an N-terminal antigen peptide sequence; or
(bi) the dimer is a heterodimer of an aAPM and a second molecule,
wherein the aAPM is a single polypeptide sequence comprising in amino-to-carboxy terminal order: an antigen-presenting domain, a dimerization domain, an immunoglobulin (Ig) Fc domain and an attachment sequence, wherein the antigen-presenting domain comprises in amino-to-carboxy terminal order: the antigen peptide sequence and an MEW class II portion, and/or
wherein the dimerization domain comprises a parallel coiled-coiled acidic or basic zipper motif.

10. A composition comprising
a plurality of aAPCs according to claim 1, wherein the composition comprises a plurality of identical aAPCs, optionally the identical aAPCs comprise a single capture molecule specific for a respective single effector molecule or the identical aAPCs comprise several capture molecules specific for several respective effector molecules.

11. An assay for determining an antigen-specific T cell response, the assay comprising the following steps:
(a) contacting T cells with a composition according to claim 10 under conditions and for a time suitable to elicit a response from the T cells upon presentation of the antigen peptide sequence by the identical aAPCs;
(b) separating the aAPCs from the T cells;
(c) contacting the aAPCs with detection antibodies against one or more effector molecules for which the one or more capture molecules of the aAPCs are specific;
(d) analysing the release of effector molecules of the T cells by detecting the effector molecules captured by the one or more capture molecules of the aAPCs,
thereby determining the T cell response specific to the antigen peptide sequence presented by the identical aAPCs.

12. An assay for determining a plurality of antigen-specific T cell responses, the assay comprising the following steps:
(a) contacting T cells with a composition according to claim 10 under conditions and for a time suitable to elicit responses from the T cells upon presentation of each of the different antigen peptide sequences presented by each group of aAPCs;
(b) separating the aAPCs from the T cells;
(c) contacting the aAPCs with detection antibodies against one or more effector molecules for which the one or more capture molecules of the aAPCs within each group of aAPCs are specific;
(d) identifying and separating the aAPCs of each of the groups of aAPCs;
(e) analysing the release of effector molecules of the T cells by detecting the effector molecules captured by the one or more capture molecules of the aAPCs within each group of aAPCs,
thereby determining a plurality of antigen-specific T cell responses specific to each antigen peptide sequence presented by each group of aAPCs.

13. The assay according to claim 11, wherein
the T cells are comprised within a fraction of peripheral blood mononuclear cells (PBMCs) or are purified T cells, and/or
the step of separating aAPCs from the T cells comprises washing the aAPCs under conditions suitable to maintain viability of the T cells and subsequently collecting the separated T cells for further in vitro cell culture.

14. A vector comprising
a polynucleotide sequence encoding the single polypeptide sequence of the aAPM as defined in claim 4.

15. The aAPC according to claim 2, wherein
the one or more capture antibodies are specific for the same effector molecule or comprise one or more groups of capture antibodies, each group being specific for a different effector molecule, and/or
the aAPC further comprises one or more co-stimulatory molecules attached to the surface for enhancing the T cell's response to presentation of the antigen peptide sequence.

16. The aAPC according to claim 15, wherein
(a) the one or more co-stimulatory antibodies are specific for the same co-stimulatory receptor, or
(b) the one or more co-stimulatory antibodies comprise one or more groups of co-stimulatory antibodies, each group being specific for a different group of the same co-stimulatory receptors.

17. The aAPC according to claim 4, wherein
(a) the antigen-presenting domain of the single polypeptide sequence comprises in amino-to-carboxy terminal order: the antigen peptide sequence, a first linker sequence, and an MHC class I portion, and/or
(b) the MHC class I portion in amino-to-carboxy terminal order comprises a β₂-microglobulin sequence, a second linker sequence, an MHC class I HLA-A*02:01α1 sequence, an MHC class I HLA-A*02:01 α2 sequence and an MHC class I HLA-A*02:01 α3 sequence.

18. The aAPC according to claim 17, wherein
the first linker sequence comprises a first cysteine residue and the MHC class I HLA-A*02:01 α1 sequence comprises a second cysteine residue, wherein the first and second cysteine residues form a disulfide trap enhancing the association of the antigen peptide sequence to the MHC class I portion of the antigen-presenting domain through covalent linkage, and/or
the first linker sequence comprises SEQ ID NO: 30.

19. The aAPC according to claim 4, wherein
(a) the MEW class I portion comprises SEQ ID NO: 31 or SEQ ID NO: 32, or
(b) the aAPM comprises in amino-to-carboxy terminal order: the antigen peptide sequence and SEQ ID NO: 33.

20. The aAPC according to claim 4, comprising a dimer comprising an aAPM, wherein the dimer is a heterodimer of the aAPM and a second molecule, and in which the aAPM is a single polypeptide sequence comprising in amino-to-carboxy terminal order: an antigen-presenting domain, a dimerization domain, an immunoglobulin (Ig) Fc domain and an attachment sequence, wherein the sequence of the antigen presenting domain of the aAPM comprises an N-terminal antigen peptide sequence and an MEW class II portion,
wherein, the MEW class II portion in amino-to-carboxy terminal order comprises an MHC class II HLA-DRβ1 sequence and an MEW class II HLA-DRβ2 sequence and wherein the second molecule comprises an MHC class II HLA-DRα1 sequence and an MHC class II HLA-DRα2 sequence, which is not tethered to an antigenic peptide sequence; or
wherein the MEW class II portion of the aAPM comprises the DRB1*03:01 sequence of SEQ ID NO: 50 and the second molecule comprises DRA*01:01 sequence of SEQ ID NO: 51.

21. The aAPC according to claim 20, wherein the aAPM comprises in amino-to-carboxy terminal order: the antigen peptide sequence, and SEQ ID NO: 55; and the second molecule comprises SEQ ID NO: 56.

22. A composition comprising a plurality of groups of aAPCs according to claim 1, wherein all aAPCs of each group are coded identically and wherein the antigen peptide sequence presented by the aAPCs of each group is identical within the group but different between each of the groups, optionally the aAPCs of each group comprise a single capture molecule specific for a respective single effector molecule, or the aAPCs of each group comprise several capture molecules specific for several respective effector molecules.

23. A vector comprising a polynucleotide sequence for polycistronic expression of both peptide chains of the dimer defined in claim 9.
